SEQUENCE LISTING (24 sequences)

SEQ ID NO: 1 - protein, 8 aa, Homo sapiens
DSAIRKYY

SEQ ID NO: 2 - protein, 7 aa, Homo sapiens
IYASGSS

SEQ ID NO: 3 - protein, 9 aa, Homo sapiens
AAITGTTDL

SEQ ID NO: 4 - DNA, 24 bp, Homo sapiens
gatagtgccatcaggaaatactat

SEQ ID NO: 5 - DNA, 21 bp, Homo sapiens
atctatgccagtgggagttct

SEQ ID NO: 6 - DNA, 27 bp, Homo sapiens
gcggccataactggaacgactgatctc

SEQ ID NO: 7 - protein, 6 aa, Homo sapiens
QGINRR

SEQ ID NO: 8 - protein, 3 aa, Homo sapiens
AVS

SEQ ID NO: 9 - protein, 9 aa, Homo sapiens
LQSNNYPLT

SEQ ID NO: 10 - DNA, 18 bp, Homo sapiens
cagggaattaacaggagg

SEQ ID NO: 11 - DNA, 9 bp, Homo sapiens
gctgtgtcc

SEQ ID NO: 12 - DNA, 27 bp, Homo sapiens
ctacagtctaacaactatcccctcact

SEQ ID NO: 13 - protein, 104 aa, Homo sapiens
QVQLQESGPGVVKPSETLSLTCSVSDSAIRKYYWSWIRQPPGQGLEYIGYIYASGSSFYNPSFKSRVSMSVDATNNQFYLKLTSVTAADTAVYYCAAITGTTDL

SEQ ID NO: 14 - protein, 97 aa, Homo sapiens
DIQMTQSPSSVSASVGDRVTITCRASQGINRRLAWYQQKPGKAPKRLIYAVSTLQSGVPSRFNGSGSGTDFTLTVNNVQPDDLAMYFCLQSNNYPLT

SEQ ID NO: 15 - DNA, 312 bp, Homo sapiens
caggtgcagctgcaggagtcgggcccaggagtggtgaagccttcggagaccctgtccctcacctgcagtgtctctgatagtgccatcaggaaatactattggagttggatccggcagcccccaggacagggactggagtatattggatatatctatgccagtgggagttctttttataatccctccttcaagagtcgagtcagcatgtcggtagacgcgaccaacaatcagttctacctgaagttgacttctgtcaccgccgcggacacggccgtatattactgtgcggccataactggaacgactgatctc

SEQ ID NO: 16 - DNA, 291 bp, Homo sapiens
gacatccagatgacccagtctccatcttctgtgtctgcttctgtaggagacagggtcaccatcacttgccgggcgagtcagggaattaacaggaggttagcctggtatcagcagaaaccagggaaagcccctaaacgcctgatctatgctgtgtccactttgcaaagtggagtcccatcccgattcaacggcagtggatctgggacagatttcactctcactgtcaataacgtgcagcctgatgatcttgcaatgtatttttgtctacagtctaacaactatcccctcact

SEQ ID NO: 17 - protein, 8 aa, Homo sapiens
IYASGSSF

SEQ ID NO: 18 - protein, 4 aa, Homo sapiens
AVST

SEQ ID NO: 19 - DNA, 24 bp, Homo sapiens
atctatgccagtgggagttctttt

SEQ ID NO: 20 - DNA, 12 bp, Homo sapiens
gctgtgtccact

SEQ ID NO: 21 - protein, 115 aa, Homo sapiens
QVQLQESGPGVVKPSETLSLTCSVSDSAIRKYYWSWIRQPPGQGLEYIGYIYASGSSFYNPSFKSRVSMSVDATNNQFYLKLTSVTAADTAVYYCAAITGTTDLWGRGTLVTVSS

SEQ ID NO: 22 - protein, 107 aa, Homo sapiens
DIQMTQSPSSVSASVGDRVTITCRASQGINRRLAWYQQKPGKAPKRLIYAVSTLQSGVPSRFNGSGSGTDFTLTVNNVQPDDLAMYFCLQSNNYPLTFGGGTKVEIK

SEQ ID NO: 23 - DNA, 346 bp, Homo sapiens
caggtgcagctgcaggagtcgggcccaggagtggtgaagccttcggagaccctgtccctcacctgcagtgtctctgatagtgccatcaggaaatactattggagttggatccggcagcccccaggacagggactggagtatattggatatatctatgccagtgggagttctttttataatccctccttcaagagtcgagtcagcatgtcggtagacgcgaccaacaatcagttctacctgaagttgacttctgtcaccgccgcggacacggccgtatattactgtgcggccataactggaacgactgatctctggggccgtggcaccctggtcactgtctcctcag

SEQ ID NO: 24 - DNA, 322 bp, Homo sapiens
gacatccagatgacccagtctccatcttctgtgtctgcttctgtaggagacagggtcaccatcacttgccgggcgagtcagggaattaacaggaggttagcctggtatcagcagaaaccagggaaagcccctaaacgcctgatctatgctgtgtccactttgcaaagtggagtcccatcccgattcaacggcagtggatctgggacagatttcactctcactgtcaataacgtgcagcctgatgatcttgcaatgtatttttgtctacagtctaacaactatcccctcactttcggcggtgggaccaaggtggagatcaaac

(19) United States
(12) Patent Application Publication
(10) Pub. No.: US 2016/0024200 A1
(43) Pub. Date: Jan. 28, 2016

(54) HUMAN MONOCLONAL ANTIBODIES THAT NEUTRALIZE BIOACTIVITY OF GRANULOCYTE MACROPHAGE COLONY-STIMULATING FACTOR AND METHODS AND USES THEREOF

(71) Applicant: John SCHRADER, Vancouver (CA)

(72) Inventor: John Schrader, Vancouver (CA)

(21) Appl. No.: 14/774,861

(22) PCT Filed: Mar. 6, 2014
(86) PCT No.: PCT/CA2014/000180

§ 371 Date: Sep. 11, 2015

Related U.S. Application Data
(60) Provisional application No. 61/782,012, filed on Mar. 14, 2013.

(51) Int. Cl. C07K 16/24

(52) U.S. Cl. CPC C07K 16/243; C07K 2317/565; C07K 2317/76; C07K 2317/92

(57) ABSTRACT

The present disclosure relates to an isolated human monoclonal antibody or antibody fragment that has a high potency for neutralizing the bioactivity of GM-CSF comprising the heavy chain CDR sequences of SEQ ID NOs: 1, 2 and 3 and the light chain CDR sequences of SEQ ID NOs: 7, 8 and 9, or functional variants thereof. The disclosure also relates to methods and uses of the antibody or antibody fragment for suppressing an immune response, heating inflammation, pain, cancer, and bone or cartilage loss.

### RELATED APPLICATIONS

This non-provisional application claims priority from U.S. provisional application No. 61/782,012 filed on Mar. 14, 2013, which is incorporated herein by reference in its entirety.

### FIELD

The present disclosure relates generally to anti-granulocyte macrophage colony stimulating factor (GM-CSF) antibodies. In particular, the present disclosure relates to human monoclonal antibodies as well as to methods and uses thereof.

### BACKGROUND

The ability to make truly human monoclonal antibodies from humans has been slow to start because generating human hybridomas or heterospecies hybridomas were very inefficient. Human antibodies have been made from infectious diseases from humans infected or vaccinated with pathogen or a pathogen antigen by the hybridoma technique (Gigliotti, 1984 and Janoff et al. United States Patent Publication 20040198960; Krause, 2012). It has also been possible to make human autoantibodies against human antigens by the hybridoma technique (Shoenfeld, 1982; Shoenfeld, 1983).

GM-CSF stimulates the proliferation and differentiation of committed progenitors that generate neutrophils, monocytes, macrophages and dendritic cells (Metcalf, 2008). GM-CSF also activates the differentiated myeloid effector cells like neutrophils, eosinophils, dendritic cells, macrophages or monocytes to increase their activity or maturation and prolong their survival (Handman, 1979; Hamilton, 1980; Lopez, 1986). GM-CSF promoted the fusion of prefusion osteoclasts to activate them to resorb bone (Lee, 2009).

Numerous investigators have proposed the use of antibodies against GM-CSF to treat inflammatory responses of macrophages and granulocyte cells (Lopez, 1986; Williamson, 1988; Zvaifler, 1988).

The GM-CSF receptor has a GM-CSF-specific subunit, termed the GM-CSF receptor alpha (GM-CSFRα) and a common subunit shared between the interleukin-3 receptor and interleukin-5 receptor, termed the f3, (Hercus, 2009). The simple model of GM-CSF binding a heterodimer of the GM-CSFRα and the β_{c} making a trimer, was more complex (Hercus, 2009). Crystallography has indicated that the signalling complex of GM-CSF and the subunits of the GM-CSFRα and the β_{c} is a dodecamer made of four GM-CSF molecules and four heterodimers of the subunits of the GM-CSF receptor (Hansen, 2008). The GM-CSF signalling dodecamer complex contains two hexamers made of two GM-CSF molecules, two GM-CSFRα molecules and two β_{c} molecules. The GM-CSF interacts in one hexamer with one GM-CSFRα, the β_{c} of the heterodimer and another β_{c} in the hexamer (Hansen, 2008). Hercus et al. (Hercus, 2009) states that there may be physical interactions between two GM-CSF molecules in the dodecamer complex. Thus there are many sites where a monoclonal antibody could bind to GM-CSF and sterically inhibit the interaction with GM-CSF and its complex, three-dimensional signalling receptor (Hansen, 2008; Hercus, 2009; Wang, Thomson, 2013).

GM-CSF and its role in inflammatory disease: Preclinical data indicates that GM-CSF has a critical role in arthritis, multiple sclerosis, asthma, chronic obstructive pulmonary disease (COPD) (Hamilton, 2008) and chronic pain (Schweizerhof, 2009; Cook, 2013). The prevalence of rheumatoid arthritis has been associated with polymorphisms in the GM-CSF gene (Okada, 2012). Antibodies that neutralize the biological activity of GM-CSF may be useful in treating inflammatory diseases like arthritis (Cook, 2001; Burmester, 2011; Plater-Zyberk, 2007). Anti-GM-CSF antibodies reduced cartilage proteoglycan depletion in a model of arthritis (Plater-Zyberk, 2007). Antibodies against GM-CSF may be useful in multiple sclerosis (McQualter, 2001; Codarri, 2011; El-Behi, 2011). Antibodies against GM-CSF also may be useful for treating ankylosing spondylitis (Sherlock, 2012) because IL-23 induces GM-CSF (Codarri, 2011; El-Behi, 2011). Further, antibodies that neutralize the biological activity of GM-CSF in a mouse model of psoriasis reduced the disease (Schon, 2000). Clinical data indicates GM-CSF was detected at sites of inflammation in rheumatoid arthritis e.g. synovial cavity in rheumatoid arthritis (Williamson, 1988; Zvaifler, 1988). Promising results have been obtained with Phase I and II trials in rheumatoid arthritis with artificial, genetically engineered antibodies from human genes against GM-CSF (Hamilton, 2013) or against the GM-CSF receptor (Burmester, 2011) and in trials in multiple sclerosis (Hamilton, 2013).

GM-CSF also plays a role in suppressing immune responses against cancer because 31% of human cancers produced GM-CSF, including renal, colon, prostate, ovarian, melanoma and cervical cancers (Bronte, 1999). Other researchers have found the same, with gliomas (Frei, 1992; Revoltella, 2012) and pancreatic cancers (Bayne, 2012; Pylayeva-Gupta, 2012). GM-CSF in mice stimulates myeloid-derived suppressor cells that suppress the adaptive T cell immune response against cancers (Serafini, 2004; Lindau, 2013). Thus monoclonal antibodies against mouse GM-CSF in mice reduced pancreatic cancer size (Bayne, 2012). Mouse GM-CSF has a very different sequence from human GM-CSF so monoclonal antibodies that bind to mouse GM-CSF do not bind to human GM-CSF.

Juvenile myelomonocytic leukemia or juvenile chronic myelogenous leukemia is characterized by a hypersensitivity to GM-CSF (Koike, 2008). In vitro, proliferation of juvenile myelomonocytic leukemia cells has been demonstrated to be inhibited specifically by anti-sera against GM-CSF (Gualtieri, 1989). Juvenile myelomonocytic leukemia has been shown to be treated by a GM-CSF antagonist, a mutant form of GM-CSF (Iversen, 1997).

Schweizerhof et al. report that GM-CSF causes sensory neurons to sprout and the GM-CSF receptor is expressed on sensory neurons and abrogation of GM-CSF signaling attenuated tumor pain (Schweizerhof, 2009).

It is known that humans with idiopathic pulmonary alveolar proteinosis (IPAP) have autoantibodies that neutralize the bioactivity of the hemopoietic growth factor or cytokine termed granulocyte-macrophage colony-stimulating factor (GM-CSF) (Tanaka, 1999; Kitamura, 1999; Uchida, 2003). There are two reports of human monoclonal auto-antibodies against GM-CSF from blood samples of patients with IPAP. One was generated with hybridoma technology (Li, 2006), however, these monoclonal antibodies only weakly neutralized the bioactivity of GM-CSF. Takada et al WO/2009/064399 transformed B cells from an IPAP patient with Epstein-Barr virus (EBV). EBV induces expression of Activation-Induced cytidine Deaminase (AID) which physiologically induces somatic mutations in the immunoglobulin genes in vitro (He, 2003; Gil, 2007; Epeldegui, 2007; Heath, 2012). The EBV-transformed B cells were in culture for 12-16 weeks before the antibodies were cloned and sequenced. AID is highly expressed in EBV-transformed B cells and would have produced randomly somatically mutated antibodies (Heath, 2012). The procedure of Takada et al with culturing in vitro for 12-16 weeks, in contrast to the germinal centre in vivo, has no antigen present in vitro to select for affinity-maturation. Also, in contrast to the germinal centre in vivo, there is no selective pressure in vitro for maintaining the thermodynamic stability of the antibodies (Wang, Sen, 2013).

### SUMMARY

The present inventor developed an authentic human monoclonal antibody against GM-CSF, termed F1, which neutralizes potently the bioactivity of GM-CSF. This human monoclonal antibody binds GM-CSF tightly with high-affinity and binds to a unique epitope that affects by steric hindrance the GM-CSF interaction with the GM-CSF receptor signaling complex. The present inventor has mapped 4 non-overlapping epitopes on GM-CSF by other human monoclonal antibodies against GM-CSF. The F1 antibody binds an epitope spanning the two non-overlapping epitopes defined by the other antibodies against GM-CSF.

Accordingly, the present disclosure provides an isolated antibody or antibody fragment that neutralizes the bioactivity of GM-CSF comprising the heavy chain CDR sequences of SEQ ID NOS:1, 2 and 3 and/or functional variants thereof or the light chain CDR sequences of SEQ ID NOS:7, 8 and 9 or functional variants thereof.

In one embodiment, the isolated antibody or antibody fragment comprises the heavy chain CDR sequences of SEQ ID NOS:1, 2 and 3 or functional variants thereof and the light chain CDR sequences of SEQ ID NOS:7, 8 and 9 or functional variants thereof.

In another embodiment, the isolated antibody further comprises a phenylalanine as the first amino acid in the framework region after the heavy chain CDR2.

In another embodiment, the isolated antibody further comprises a threonine as the first amino acid in the framework region after the light chain CDR2.

In another embodiment, the present disclosure provides an isolated antibody or antibody fragment that neutralizes the bioactivity of GM-CSF comprising the heavy chain variable region as shown in the amino acid sequence of SEQ ID NO:13 or 21 or a functional variant thereof and/or the light chain variable region as shown in the amino acid sequence of SEQ ID NO:14 or 22 or a functional variant thereof. In one embodiment, the isolated antibody or antibody fragment comprises the heavy chain variable region as shown in the amino acid sequence of SEQ ID NO:13 or 21 or a functional variant thereof and the light chain variable region as shown in the amino acid sequence of SEQ ID NO:14 or 22 or a functional variant thereof.

In one embodiment, the antibody fragment is a Fab, Fab′, F(ab′)2, Fv or scFv.

In another embodiment, the isolated antibody or antibody fragment thereof inhibits GM-CSF in the TF-1 assay with an IC50 of less than 3 ng/mL. In another embodiment, the isolated antibody or antibody fragment thereof inhibits GM-CSF in the TF-1 assay with an IC50 of less than 6 ng/mL. In another embodiment, the isolated antibody or antibody fragment thereof inhibits GM-CSF in the TF-1 assay with an IC50 of less than 9 ng/mL. In yet another embodiment, the antibody or antibody fragment competes for binding to GM-CSF with an antibody having the heavy chain variable region as shown in SEQ ID NO:13 or 21 and the light chain variable region as shown in SEQ ID NO:14 or 22.

Also disclosed herein are isolated nucleic acid molecules encoding the isolated antibody or antibody fragment thereof disclosed herein, expression vectors comprising the nucleic acid molecules disclosed herein operatively linked to suitable regulatory sequences and host cells comprises said nucleic acid molecules or expression vectors.

The present disclosure further provides a pharmaceutical composition comprising the antibody or antibody fragment of the present disclosure, the nucleic acid molecules encoding the isolated heavy and/or light chain of the present disclosure, the expression vectors expressing the isolated heavy and/or light chain of the present disclosure or the host cell of the present disclosure, and a pharmaceutically acceptable carrier.

The disclosure further provides methods and uses of suppressing an immune response, such as an innate or adaptive response. Accordingly, in one embodiment, there is provided a method of suppressing an immune response in a subject comprising administering an antibody or antibody fragment of the present disclosure, expression vectors expressing the isolated heavy and light chain of the present disclosure, a host cell of the present disclosure or a pharmaceutical composition of the present disclosure to the subject in need thereof. In another embodiment, there is provided a use of an antibody or antibody fragment of the present disclosure, expression vectors expressing the isolated heavy and light chain of the present disclosure, a host cell of the present disclosure or a pharmaceutical composition of the present disclosure for suppressing an immune response in a subject in need thereof. Further provided is a use of an antibody or antibody fragment of the present disclosure, expression vectors expressing the isolated heavy and light chain of the present disclosure, a host cell of the present disclosure or a pharmaceutical composition of the present disclosure in the preparation of a medicament for suppressing an immune response in a subject in need thereof. Even further provided is an antibody or antibody fragment of the present disclosure, expression vectors expressing the isolated heavy and light chain of the present disclosure, a host cell of the present disclosure or a pharmaceutical composition of the present disclosure for use in suppressing an immune response in a subject in need thereof.

In one embodiment, the methods and uses are for treating inflammation or an inflammatory disease. In an embodiment, the inflammatory disease is arthritis, multiple sclerosis, asthma, chronic obstructive pulmonary disease (COPD), ankylosing spondylitis, or psoriasis.

In one embodiment, the methods and uses are for treating pain caused by cancer or osteoarthritis.

In another embodiment, the methods and uses are for treating cancer associated with myeloid-derived suppressor cells that suppress the adaptive immune response against cancer cells.

In one embodiment, the cancer is renal, colon, prostate, ovarian, melanoma, glioma, pancreatic and cervical cancers.

The disclosure further provides methods and uses of treating bone or cartilage loss, pain and/or juvenile myelomonocytic leukemia, all of which are adversely affected by GM-CSF. Accordingly, in one embodiment, there is provided a method of treating bone or cartilage loss, pain and/or juvenile myelomonocytic leukemia in a subject comprising administering an antibody or antibody fragment of the present disclosure, expression vectors expressing the isolated heavy and light chain of the present disclosure, a host cell of the present disclosure or a pharmaceutical composition of the present disclosure to the subject in need thereof. In another embodiment, there is provided a use of an antibody or antibody fragment of the present disclosure, expression vectors expressing the isolated heavy and light chain of the present disclosure, a host cell of the present disclosure or a pharmaceutical composition of the present disclosure for treating bone or cartilage loss, pain and/or juvenile myelomonocytic leukemia in a subject in need thereof. Further provided is a use of an antibody or antibody fragment of the present disclosure, expression vectors expressing the isolated heavy and light chain of the present disclosure, a host cell of the present disclosure or a pharmaceutical composition of the present disclosure in the preparation of a medicament for treating bone or cartilage loss, pain and/or juvenile myelomonocytic leukemia in a subject in need thereof. Even further provided is an antibody or antibody fragment of the present disclosure, expression vectors expressing the isolated heavy and light chain of the present disclosure, a host cell of the present disclosure or a pharmaceutical composition of the present disclosure for use in treating bone or cartilage loss, pain and/or juvenile myelomonocytic leukemia in a subject in need thereof.

Other features and advantages of the present disclosure will become apparent from the following detailed description. It should be understood, however, that the detailed description and the specific examples while indicating embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows fluorescent-activated cell sorting of GM-CSF specific memory cells. PBMC were prepared and frozen, then subsequently thawed and stained with GM-CSF conjugated with Alexa-647 and fluorochrome-labeled antibodies and Annexin V as described. Single lymphocytes were gated on forward scatter and side scatter (P2, top right panel). A gate, (P3, bottom left panel) was made by positive selection on anti-CD27 phycoerythrin and negative selection on anti-CD3-FITC (T lymphocytes), anti-IgM-FITC (IgM memory B cells) and Annexin V-FITC (apoptotic cells). Shown (P4, bottom right panel) is the GM-CSF-binding CD19-positive and CD27-positive memory B cells on the upper-right quadrant. In the right lower panel, on the X axis is the anti-CD19 APC-H7 and on the Y axis, GM-CSF conjugated with Alexa-647.

FIG. 2 shows the high potency of purified F1 (sequences of this antibody are shown in Table 2) to neutralize the bioactivity of GM-CSF in a TF-1 assay. Purified F1 was serially diluted and added to GM-CSF at a final concentration of 400 pg/mL and pre-incubated 1 hour at 37° C. Then were added an equal volume of washed TF-1 cells, at 1000 cells per well, at a final concentration of GM-CSF of 200 pg/mL and the plates were incubated for 4 days at 37° C. WST-1 was added and the incubation continued for 4 hours. The absorbance values at wavelength 450 nm, with reference wavelength of 690 nm values subtracted, were read by a plate-reader.

FIG. 3 shows that purified F1 neutralized the bioactivity of high concentrations of GM-CSF on normal human neutrophils to activate neutrophils and upregulate CD11b.

FIG. 4 shows that F1 binds to a conformational epitope. After GM-CSF was reduced with DTT and boiled with SDS, F1 did not bind to denatured, unfolded GM-CSF in an immunoblot. The control monoclonal antibody that binds to a linear epitope the His-tag, binds to a His tag on the denatured, reduced recombinant GM-CSF. If GM-CSF was not reduced with DTT, it was recognized by F1.

FIG. 5 shows that F1 binds to a unique epitope on GM-CSF not shared by the other human monoclonal antibodies generated against GM-CSF from donors with IPAP. They are named A1, A2, B1, C2, C3, C5, E1, E2, E3, E5, E6 and E7. On FIGS. 5 A and B, shown is the anti-GM-CSF monoclonal antibody attached to the chip, in A or B, respectively C3 and A1. The time trace is started when the GM-CSF flows over the chip and the surface plasmon resonance (SPR) signal is expressed in response units (RU) rises as the antibody captures the GM-CSF. When the second anti-GM-CSF monoclonal antibody is flowed over the chip (in A or B, respectively F1 and C3), the RU rises or does not depending on whether the second anti-GM-CSF monoclonal antibody binds to the GM-CSF captured by the first anti-GM-CSF monoclonal antibody attached to the chip. Then the third anti-GM-CSF monoclonal antibody was flowed over the chip (in A or B, respectively E6 and B1) the increase in RU indicated that it bound to GM-CSF. C, shown is a summary of the epitope mapping experiments with an “X” meaning that the two anti-GM-CSF monoclonal antibodies cannot bind simultaneously to a single molecule of GM-CSF and an “&” meaning they can bind simultaneously to GM-CSF. D, shown is a diagram drawn from data in C that demonstrated the F1 bound to an epitope that spans two non-overlapping epitopes binding in contrast to the other human monoclonal antibodies against GM-CSF.

FIG. 6 shows synergy of two human monoclonal antibodies against GM-CSF, C3 and C5 in neutralizing the bioactivity of GM-CSF in the TF-1 assay. Note that C3 and C5 are less potent at neutralizing the bioactivity of GM-CSF than F1 (upper panel). In the same experiment, shown is a titration of an equal mixture of C3 and C5 (lower panel). For ease of comparison the F1 titration graph is repeated in the lower panel. C3 and C5 bind to non-overlapping epitopes (FIG. 5C) and the F1 epitope spans the C3 epitope and the C5 epitope. F1 is titrated in the TF-1 assay and has an equal IC50 as an equal mixture of C3 and C5.

### DETAILED DESCRIPTION

The inventor has obtained a highly potent human monoclonal antibody against granulocyte-macrophage colony-stimulating factor (GM-CSF) termed the “F1 antibody” and has determined the sequence of the light and heavy chain variable regions and complementarity determining regions (CDRs) 1, 2 and 3 of the antibody (Table 2).

The term “GM-CSF” as used herein refers to granulocyte macrophage colony stimulating factor from any species or source and includes the full-length protein as well as fragments or portions of the protein. The Genbank accession number for human GM-CSF is GI:371502128.

In an embodiment, the disclosure provides isolated heavy chain complementarity determining region 1 (CDR1) comprising the amino acid sequence DSAIRKYY (SEQ ID NO:1) or encoded by the nucleic acid sequence as shown in SEQ ID NO:4; isolated heavy chain complementarity determining region 2 (CDR2) comprising the amino acid sequence IYASGSS (SEQ ID NO:2) or encoded by the nucleic acid sequence as shown in SEQ ID NO:5; isolated heavy chain complementarity determining region 3 (CDR3) comprising the amino acid sequence AAITGTTDL (SEQ ID NO:3) or encoded by the nucleic acid sequence as shown in SEQ ID NO:6; isolated light chain CDR1 comprising the amino acid sequence QGINRR (SEQ ID NO:7) or encoded by the nucleic acid sequence as shown in SEQ ID NO:10; isolated light chain CDR2 comprising the amino acid sequence AVS (SEQ ID NO:8) or encoded by the nucleic acid sequence as shown in SEQ ID NO:11; and isolated light chain CDR3 comprising the amino acid sequence LQSNNYPLT (SEQ ID NO:9) or encoded by the nucleic acid sequence as shown in SEQ ID NO:11.

The term “heavy chain complementarity determining region” as used herein refers to regions of hypervariability within the heavy chain variable region of an antibody molecule. The heavy chain variable region has three complementarity determining regions termed heavy chain complementarity determining region 1, heavy chain complementarity determining region 2 and heavy chain complementarity determining region 3 from the amino terminus to carboxy terminus, for example, as defined by IMGT (Brochet, 2008; Giudicelli, 2011).

The term “light chain complementarity determining region” as used herein refers to regions of hypervariability within the light chain variable region of an antibody molecule. Light chain variable regions have three complementarity determining regions termed light chain complementarity determining region 1, light chain complementarity determining region 2 and light chain complementarity determining region 3 from the amino terminus to the carboxy terminus, for example, as defined by IMGT (Brochet, 2008; Giudicelli, 2011).

Also provided are isolated heavy chain variable regions comprising the heavy chain CDR1, CDR2 and/or CDR3 disclosed herein and isolated light chain variable regions comprising the light chain CDR1, CDR2 and/or CDR3 disclosed herein.

The term “heavy chain variable region” as used herein refers to the variable region of a heavy chain.

The term “light chain variable region” as used herein refers to the variable region of a light chain.

The present inventor also found somatic mutations in the framework region (FR3) following CDR2 of the H and L chains. Accordingly, in another embodiment, the heavy chain variable region comprises a phenylalanine as the first amino acid after the CDR2 of the heavy chain disclosed herein and/or light chain variable region comprises a threonine as the first amino acid after the CDR2 of the light chain disclosed herein.

In an embodiment, the heavy chain variable region comprises the amino acid sequence shown in SEQ ID NO:13 or is encoded by the nucleic acid sequence as shown in SEQ ID NO:15.

In another embodiment, the light chain variable region comprises the amino acid sequence shown in SEQ ID NO:14 or is encoded by the nucleic acid sequence as shown in SEQ ID NO:16.

SEQ ID NOs: 13 and 14 include the amino acid sequence of all three CDR's of the heavy chain variable and the light chain variable region but do not include the conserved portion of the framework 4 region (FR4) encoded by the J gene. A person skilled in the art of IGHJ and IGKJ genes and amplifying the variable regions with primers by PCR can readily express an authentic human monoclonal antibody with this specificity against GM-CSF with SEQ ID NOs: 13 and 14.

Accordingly, in another embodiment, the heavy chain variable region comprises the amino acid sequence as shown in SEQ ID NO:21 or is encoded by the nucleic acid sequence as shown in SEQ ID NO:23.

In another embodiment, the light chain variable region comprises the amino acid sequence as shown SEQ ID NO:22 or is encoded by the nucleic acid sequence as shown in SEQ ID NO:24.

The disclosure further provides an isolated antibody or antibody fragment that neutralizes the bioactivity of GM-CSF, wherein the antibody comprises at least one heavy chain complementarity determining region as shown in SEQ ID NOs:1-3 and/or at least one light chain complementarity determining region as shown in SEQ ID NOs:7-9.

The phrase “that neutralizes the bioactivity of GM-CSF” as used herein refers to the steric interference of the antibody by binding to an epitope of GM-CSF to prevent the functional signaling complex formed by GM-CSF with the GM-CSF receptor. Convenient assays that measure the bioactivity of GM-CSF and the signaling made by GM-CSF can be made on appropriate cells that express the GM-CSF receptor. For example, the TF-1 human leukemia cell line and assays of GM-CSF bioactivity can include activating JAK2 kinase, (for example by immunobloting with Phospho-Jak2 (Tyr221) sold by Cell Signaling, #3774S), phosphorylating STATS, (for example measured by flow cytometry with antibody that binds to phospho-Stat5, sold by Cell Signaling #4322) or inducing signals that promote survival or growth. Another example is primary neutrophils obtained from healthy humans and assays of GM-CSF bioactivity include inducing higher levels of expression CD11b, prolonging survival and polarization of shape which can be measured on by FACS (Lopez, 1986).

In one embodiment, the isolated antibody or antibody fragment that neutralizes the bioactivity of GM-CSF comprises the heavy chain CDRs comprising the amino acid sequence of SEQ ID NOS:1, 2 and 3 and/or the light chain CDRs comprising the amino acid sequences of SEQ ID NOS:7, 8 and 9. In another embodiment, the isolated antibody or antibody fragment that neutralizes the bioactivity of GM-CSF comprises the heavy chain CDRs as shown in SEQ ID NOs:1, 2, and 3 and the light chain CDRs as shown in SEQ ID NOs:7, 8 and 9.

In another embodiment, the isolated antibody or antibody fragment further comprises a phenylalanine as the first amino acid after the CDR2 of the heavy chain (see SEQ ID NO:17 for CDR2 plus this amino acid). In yet another embodiment, the isolated antibody or antibody fragment further comprises a threonine as the first amino acid after the CDR2 of the light chain (see SEQ ID NO:18 for CDR2 plus this amino acid).

In another embodiment, the isolated antibody or antibody fragment that neutralizes the bioactivity of GM-CSF comprises the amino acid sequence of SEQ ID NO:13 (heavy chain variable region excluding framework 4 region) or SEQ ID NO:21 (heavy chain variable region including framework 4 region) and/or the amino acid sequence of SEQ ID NO: 14 (light chain variable region excluding framework 4 region) or SEQ ID NO:22 (light chain variable region including framework 4 region). In yet another embodiment, the isolated antibody or antibody fragment that neutralizes the bioactivity of GM-CSF comprises, or consists, of the amino acid sequence of SEQ ID NO:13 (heavy chain variable region excluding framework 4 region) or SEQ ID NO:21 (heavy chain variable region including framework 4 region), and the amino acid sequence of SEQ ID NO: 14 (light chain variable region excluding framework 4 region) or SEQ ID NO:22 (light chain variable region including framework 4 region).

The disclosure also provides variants of the CDR sequences, light chain and heavy chain variable sequences and antibodies comprising said variant sequences. Such variants include proteins that perform substantially the same function as the specific proteins or fragments disclosed herein in substantially the same way. For example, a functional variant of a CDR or light chain or heavy chain variable region or antibody will be able to bind to an antigen or epitope recognized by the native CDR or light chain or heavy chain variable region or antibody.

In one embodiment, the variant amino acid sequences of the light chain CDR1, CDR2 and CDR3, and the heavy chain CDR1, CDR2 and CDR3 have at least 50%, at least 60%, at least 70%, at least 80%, at least 90%, at least 95% or at least 98% sequence identity to the CDR sequences disclosed herein.

In another embodiment, the variant amino acid sequences of the light chain variable region and the heavy chain variable region have at least 50%, at least 60%, at least 70%, at least 80%, at least 90%, at least 95% or at least 98% sequence identity to the light chain variable region and heavy chain variable region sequences disclosed herein.

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. An optional, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present disclosure. BLAST protein searches can be performed with the XBLAST program parameters set, e.g., to score-50, wordlength=3 to obtain amino acid sequences homologous to a protein molecule of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search, which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another optional, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In an embodiment, the isolated antibody or antibody fragment or variants thereof inhibit GM-CSF in the TF-1 assay with an 1050 of less than 3 ng/mL. In another embodiment, the isolated antibody or antibody fragment or variants thereof inhibit GM-CSF in the TF-1 assay with an IC50 of less than 6 ng/mL. In yet another embodiment, the isolated antibody or antibody fragment or variants thereof inhibit GM-CSF in the TF-1 assay with an IC50 of less than 9 ng/mL.

In a further embodiment, the isolated antibody or antibody fragment or variants thereof inhibit the TF-1 assay with an IC50 of less than 3 ng/mL and compete with binding to GM-CSF of an antibody comprising the heavy chain variable region as shown in SEQ ID NO:13 or 21 and the light chain variable region as shown in SEQ ID NO:14 or 22. In another embodiment, the isolated antibody or antibody fragment or variants thereof inhibit the TF-1 assay with an IC50 of less than 6 ng/mL and compete with binding to GM-CSF of an antibody comprising the heavy chain variable region as shown in SEQ ID NO:13 or 21 and the light chain variable region as shown in SEQ ID NO:14 or 22. In yet another embodiment, the isolated antibody or antibody fragment or variants thereof inhibit the TF-1 assay with an IC50 of less than 9 ng/mL and compete with binding to GM-CSF of an antibody comprising the heavy chain variable region as shown in SEQ ID NO:13 or 21 and the light chain variable region as shown in SEQ ID NO:14 or 22.

In another embodiment, the present disclosure provides an isolated antibody that competes with the F1 antibody for binding to the same or similar epitope of GM-CSF. Determining whether a test antibody can compete with a given antibody can be readily done by a person skilled in the art. For example, to test whether another monoclonal antibody (X) binds to the same epitope as F1, a surface plasmon resonance experiment can be done, with F1 attached to the chip or X attached to the chip. The GM-CSF should bind to F1 or X and the other antibody, X or F1, being flowed over as analyte to confirm the epitope. If X binds simultaneously with F1 to GM-CSF, X binds to a different non-overlapping epitope on GM-CSF than F1 binds and thus does not compete for binding to GM-CSF with the F1 antibody. If X and F1 do not bind simultaneously to GM-CSF, X must compete with the F1 epitope. Another way to test whether another monoclonal antibody competes with F1 for the same epitope, a competitive ELISA can be done. If X competes with F1 epitope, the inhibition of binding of F1 to GM-CSF by X can be measured in a GM-CSF ELISA by labeling F1 with biotin, and detecting inhibition of binding of F1 to GM-CSF with streptavidin-conjugated alkaline phosphatase (Thomson, 2012).

The term “amino acid” includes all of the naturally occurring amino acids as well as modified amino acids.

The term “isolated polypeptides” refers to a polypeptide substantially free of cellular material or culture medium when produced by recombinant DNA techniques, or chemical precursors or other chemicals when chemically synthesized.

The term “antibody” as used herein is intended to include monoclonal antibodies, polyclonal antibodies, and chimeric antibodies, most suitably human monoclonal antibodies. The antibody may be from recombinant sources and/or produced in transgenic animals and/or isolated from blood samples of humans with idiopathic pulmonary alveolar proteinosis (IPAP).

The term “antibody fragment” as used herein is intended to include without limitations Fab, Fab′, F(ab′)2, scFv, dsFv, ds-scFv, dimers, minibodies, diabodies, and multimers thereof, multispecific antibody fragments and Domain Antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

The antibody or antibody fragments described herein also include functional variants of the sequences that inhibit the bioactivity of GM-CSF and sterically hinder the formation of functional signaling complex with the GM-CSF receptor.

In certain embodiments, the antibody or antibody fragment comprises all or a portion of a heavy chain constant region, such as an IgG1, IgG2, IgG3, IgG4, IgA1, IgA2, IgE, IgM or IgD constant region. In one embodiment, the heavy chain constant region is an IgG1 heavy chain constant region. Furthermore, the antibody or antibody fragment can comprise all or a portion of a kappa light chain constant region or a lambda light chain constant region. In one embodiment, the light chain constant region is a kappa light chain constant region. In one embodiment, the heavy and light chain constant regions are different from the heavy and light chain constant region of the antibody from which the sequences were derived.

As described herein, to produce human monoclonal antibodies, antibody producing cells (lymphocytes) can be harvested from a human sample with IPAP and then used to make monoclonal antibodies. For example they can be fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121:140-67 (1986)), and screening of combinatorial antibody libraries (Huse et al., Science 246:1275 (1989)). Alternatively methods that copy the genes encoding the antibodies produced by individual B cells for example the selected lymphocyte antibody method (Babcook et al 1996) may be used. These methods can be used to screen for monoclonal antibodies that specifically react with GM-CSF.

Recombinant human monoclonal antibodies can be cloned using the methods known in the art, for example, the methods detailed in PCT patent publication WO2007/003041.

Specific antibodies, or antibody fragments, reactive against GM-CSF, may also be generated by screening expression libraries encoding immunoglobulin genes, or portions thereof, expressed in bacteria with cell surface components. For example, complete Fab fragments, VH regions and FV regions can be expressed in bacteria using phage expression libraries (See for example Ward et al., Nature 341:544-546 (1989); Huse et al., Science 246:1275-1281 (1989); and McCafferty et al., Nature 348:552-554 (1990)).

The term “variant” as used herein includes modifications or chemical equivalents of the amino acid and nucleic acid sequences disclosed herein that perform substantially the same function as the polypeptides or nucleic acid molecules disclosed herein in substantially the same way. For example, variants of polypeptides disclosed herein include, without limitation, conservative amino acid substitutions. Variants of polypeptides also include additions and deletions to the polypeptide sequences disclosed herein. In addition, variant sequences include analogs and derivatives thereof.

Analogs of the peptides as described herein, may include, but are not limited to an amino acid sequence containing one or more amino acid substitutions, insertions, and/or deletions. Amino acid substitutions may be of a conserved or non-conserved nature. Conserved amino acid substitutions involve replacing one or more amino acids of the proteins of the disclosure with amino acids of similar charge, size, and/or hydrophobicity characteristics. When only conserved substitutions are made the resulting analog should be functionally equivalent. Non-conserved substitutions involve replacing one or more amino acids of the amino acid sequence with one or more amino acids which possess dissimilar charge, size, and/or hydrophobicity characteristics.

Without the intention of being limited thereby, in one embodiment, the substitutions of amino acids are made that preserve the structure responsible for the ability to bind and neutralize GM-CSF as disclosed herein. Conservative substitutions are described in the patent literature, as for example, in U.S. Pat. No. 5,264,558. It is thus expected, for example, that interchange among non-polar aliphatic neutral amino acids, glycine, alanine, proline, valine and isoleucine, would be possible. Likewise, substitutions among the polar aliphatic neutral amino acids, serine, threonine, methionine, asparagine and glutamine could possibly be made. Substitutions among the charged acidic amino acids, aspartic acid and glutamic acid, could probably be made, as could substitutions among the charged basic amino acids, lysine and arginine. Substitutions among the aromatic amino acids, including phenylalanine, histidine, tryptophan and tyrosine would also likely be possible. Other substitutions might well be possible.

One or more amino acid insertions may be introduced into the amino acid sequences disclosed herein. Amino acid insertions may consist of single amino acid residues or sequential amino acids ranging from 2 to 15 amino acids in length. Such variant amino acid molecules can be readily tested for neutralizing the bioactivity of GM-CSF.

Deletions may consist of the removal of one or more amino acids, or discrete portions from the amino acid sequences disclosed herein. The deleted amino acids may or may not be contiguous. The lower limit length of the resulting analog with a deletion mutation is at least 6, 7 or 8 amino acids.

Exemplary methods of making the alterations set forth above are disclosed by Sambrook et al (Molecular Cloning: A Laboratory Manual, 2nd Ed., Cold Spring Harbor Laboratory Press, 1989).

Within the context of the present disclosure, a protein of the disclosure may include various structural forms of the primary protein which retain biological activity. For example, a protein of the disclosure may be in the form of acidic or basic salts or in neutral form. In addition, individual amino acid residues may be modified by oxidation or reduction.

The disclosure also includes an isolated nucleic acid sequences that encode the antibodies and antibody fragments disclosed herein.

Further provided herein is an isolated nucleic acid sequence encoding the heavy chain complementarity determining region 1 (CDR1) comprising the nucleic acid sequence as shown in SEQ ID NO:4; an isolated nucleic acid sequence encoding the heavy chain complementarity determining region 2 (CDR2) comprising the comprising the nucleic acid sequence as shown in SEQ ID NO:5; and an isolated nucleic acid sequence encoding the heavy chain complementarity determining region 3 (CDR3) comprising the nucleic acid sequence as shown in SEQ ID NO:6; an isolated nucleic acid sequence encoding the light chain CDR1 comprising the nucleic acid sequence as shown in SEQ ID NO:10; an isolated nucleic acid sequence encoding the light chain CDR2 comprising the nucleic acid sequence as shown in SEQ ID NO:11; or an isolated nucleic acid sequence encoding the light chain CDR3 comprising the nucleic acid sequence as shown in SEQ ID NO:12.

In one embodiment, the heavy chain CDR2 nucleic acid further comprises an additional codon at the C-terminal end coding for the first framework region amino acid phenylalanine. In an embodiment, the isolated nucleic acid sequence encodes the amino acid sequence as shown in SEQ ID NO:17. In another embodiment, the light chain CDR2 nucleic acid further comprises an additional codon at the C-terminal end coding for the first framework region amino acid threonine. In an embodiment, the isolated nucleic acid sequence encodes the amino acid sequence as shown in SEQ ID NO:18. In another embodiment, the heavy chain CDR2 nucleic acid comprising the additional codon at the C-terminal end coding for the first framework region amino acid phenylalanine comprises the nucleic acid sequence as shown in SEQ ID NO:19. In another embodiment, the light chain CDR2 nucleic acid comprising the additional codon at the C-terminal end coding for the first framework region amino acid threonine comprises the nucleic acid sequence as shown in SEQ ID NO:20.

The disclosure also includes an isolated nucleic acid sequence encoding the heavy chain variable region comprising the nucleic acid sequence as shown in SEQ ID NO:15 or 23, and/or an isolated nucleic acid sequence encoding the light chain variable region comprising the nucleic acid sequence as shown in SEQ ID NO:16 or 24.

The term “nucleic acid sequence” as used herein refers to a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present disclosure may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine.

The term “isolated nucleic acid sequences” as used herein refers to a nucleic acid substantially free of cellular material or culture medium when produced by recombinant DNA techniques, or chemical precursors, or other chemicals when chemically synthesized. An isolated nucleic acid is also substantially free of sequences, which naturally flank the nucleic acid (i.e. sequences located at the 5′ and 3′ ends of the nucleic acid) from which the nucleic acid is derived. The term “nucleic acid” is intended to include DNA and RNA and can be either double stranded or single stranded, and represents the sense or antisense strand. Further, the term “nucleic acid” includes the complementary nucleic acid sequences.

The disclosure also provides isolated nucleic acid sequences encoding variants of the CDR sequences and variable region sequences discussed above.

Variant nucleic acid sequences include nucleic acid sequences that hybridize to the nucleic acid sequences encoding the amino acid sequences disclosed herein under at least moderately stringent hybridization conditions, or have at least 50%, 60%, 70%, 80%, 90%, 95% or 98% sequence identity to the nucleic acid sequences that encode the amino acid sequences disclosed herein.

The term “sequence that hybridizes” means a nucleic acid sequence that can hybridize to a nucleic acid sequence disclosed herein under stringent hybridization conditions. Appropriate “stringent hybridization conditions” which promote DNA hybridization are known to those skilled in the art, or may be found in Current Protocols in Molecular Biology, John Wiley & Sons, N.Y. (1989), 6.3.1-6.3.6. The term “stringent hybridization conditions” as used herein means that conditions are selected which promote selective hybridization between two complementary nucleic acid molecules in solution. Hybridization may occur to all or a portion of a nucleic acid sequence molecule. The hybridizing portion is at least 50% the length with respect to one of the polynucleotide sequences encoding a polypeptide. In this regard, the stability of a nucleic acid duplex, or hybrids, is determined by the Tm, which in sodium containing buffers is a function of the sodium ion concentration, G/C content of labeled nucleic acid, length of nucleic acid probe (I), and temperature (Tm=81.5° C.-16.6 (Log 10 [Na+])+0.41(%(G+C)−600/l). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a greater than 95% identity, the final wash will be reduced by 5° C. Based on these considerations stringent hybridization conditions shall be defined as: hybridization at 5× sodium chloride/sodium citrate (SSC)/5×Denhardt's solution/1.0% SDS at Tm (based on the above equation)−5° C., followed by a wash of 0.2×SSC/0.1% SDS at 60° C.

One example of a nucleic acid modification to prepare an analog is to replace one of the naturally occurring bases (i.e. adenine, guanine, cytosine or thymidine) of the sequence with a modified base such as xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8 amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Another example of a modification is to include modified phosphorous or oxygen heteroatoms in the phosphate backbone, short chain alkyl or cycloalkyl intersugar linkages or short chain heteroatomic or heterocyclic intersugar linkages in the nucleic acid molecules. For example, the nucleic acid sequences may contain phosphorothioates, phosphotriesters, methyl phosphonates, and phosphorodithioates.

A further example of an analog of a nucleic acid molecule of the disclosure is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogs have been shown to be resistant to degradation by enzymes and to have extended lives in vivo and in vitro. PNAs also bind stronger to a complimentary DNA sequence due to the lack of charge repulsion between the PNA strand and the DNA strand. Other nucleic acid analogs may contain nucleotides containing polymer backbones, cyclic backbones, or acyclic backbones. For example, the nucleotides may have morpholino backbone structures (U.S. Pat. No. 5,034,506). The analogs may also contain groups such as reporter groups, a group for improving the pharmacokinetic or pharmacodynamic properties of nucleic acid sequence.

A person skilled in the art will appreciate that the proteins of the disclosure, such as an antibody or antibody fragment disclosed herein, may be prepared in any of several ways, including, without limitation, by using recombinant methods.

Accordingly, the nucleic acid molecules encoding an antibody or antibody fragment of the disclosure may be incorporated in a known manner into an appropriate expression vector or vectors which ensures good expression of the proteins. Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule of the application and regulatory sequences selected on the basis of the host cells to be used for expression, which is operatively linked to the nucleic acid molecule. Operatively linked is intended to mean that the nucleic acid is linked to regulatory sequences in a manner which allows expression of the nucleic acid.

The disclosure therefore contemplates a recombinant expression vector containing a nucleic acid molecule(s) encoding an antibody or antibody fragment disclosed herein, and the necessary regulatory sequences for the transcription and translation of the inserted protein-sequence.

Suitable regulatory sequences may be derived from a variety of sources, including bacterial, fungal, viral, mammalian, or insect genes (For example, see the regulatory sequences described in Goeddel, Gene Expression Technology: Methods in Enzymology 185, Academic Press, San Diego, Calif. (1990)). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

The recombinant expression vectors of the disclosure may also contain a selectable marker gene which facilitates the selection of host cells transformed or transfected with a recombinant molecule of the application. Examples of selectable marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin, optionally IgG. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of recombinant expression vectors of the application and in particular to determine the effect of a mutation on expression and phenotype. It will be appreciated that selectable markers can be introduced on a separate vector from the nucleic acid of interest.

The recombinant expression vectors may also contain genes which encode a fusion moiety which provides increased expression of the recombinant protein; increased solubility of the recombinant protein; and aid in the purification of the target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMal (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Recombinant expression vectors can be introduced into host cells to produce a transformed host cell. The terms “transformed with”, “transfected with”, “transformation” and “transfection” are intended to encompass introduction of nucleic acid (e.g. a vector) into a cell by one of many possible techniques known in the art. The term “transformed host cell” as used herein is intended to also include cells capable of glycosylation that have been transformed with a recombinant expression vector of the invention. Prokaryotic cells can be transformed with nucleic acid by, for example, electroporation or calcium-chloride mediated transformation. For example, nucleic acid can be introduced into mammalian cells via conventional techniques such as calcium phosphate or calcium chloride co-precipitation, DEAE-dextran mediated transfection, lipofectin, electroporation or microinjection. Suitable methods for transforming and transfecting host cells can be found in Sambrook et al. (Molecular Cloning: A Laboratory Manual, 3rd Edition, Cold Spring Harbor Laboratory Press, 2001), and other laboratory textbooks.

Suitable host cells include a wide variety of eukaryotic host cells and prokaryotic cells. For example, the proteins of the disclosure may be expressed in yeast cells or mammalian cells. Other suitable host cells can be found in Goeddel, Gene Expression Technology: Methods in Enzymology 185, Academic Press, San Diego, Calif. (1991). In addition, the proteins of the disclosure may be expressed in prokaryotic cells, such as Escherichia coli (Zhang et al., Science 303(5656): 371-3 (2004)). In addition, a Pseudomonas based expression system such as Pseudomonas fluorescens can be used (US Patent Application Publication No. US 2005/0186666, Schneider, Jane C et al.).

The disclosure also provides compositions comprising the CDRs in an appropriate framework, variable regions and/or antibodies disclosed herein with a pharmaceutically acceptable excipient, carrier, buffer or stabilizer.

Suitable pharmaceutically acceptable carriers include essentially chemically inert and nontoxic materials that do not interfere with the effectiveness of the biological activity of the pharmaceutical composition. Suitable vehicles are described, for example, in Remington's Pharmaceutical Sciences (Remington's Pharmaceutical Sciences, 20th ed., Mack Publishing Company, Easton, Pa., USA, 2000). Examples of suitable pharmaceutical carriers include, but are not limited to, water, saline solutions, glycerol solutions, ethanol, N-(1(2,3-dioleyloxy)propyl)N,N, N-trimethylammonium chloride (DOTMA), diolesylphosphotidyl-ethanolamine (DOPE), and liposomes. Such compositions contain a therapeutically effective amount of the agent, together with a suitable amount of carrier so as to provide the form for direct administration to the patient.

In some embodiments, the composition is formulated for administration to a subject such as a human. In particular embodiments, the composition is formulated for intravenous or oral administration. Optionally, the composition is formulated for inhalative, rectal or parenteral administration, including dermal, intradermal, intragastral, intracutan, intravasal, intravenous, intramuscular, intraperitoneal, intranasal, intravaginal, intrabuccal, percutaneous, subcutaneous, sublingual, topical or transdermal administration.

Further provided herein are methods and uses of the CDRs in an appropriate framework, variable regions, antibodies or antibody fragments thereof and/or compositions disclosed herein for suppressing an immune response in a subject. In one embodiment, the disclosure provides a method for suppressing an immune response in a subject comprising administering the CDRs in an appropriate framework, variable regions, antibodies or antibody fragments thereof and/or compositions described herein to a subject in need thereof. Also provided is use of the CDRs in an appropriate framework, variable regions, antibodies or antibody fragments thereof and/or compositions described herein for suppressing an immune response in a subject in need thereof. Further provided is use of the CDRs in an appropriate framework, variable regions, antibodies or antibody fragments thereof and/or compositions described herein for preparing a medicament for suppressing an immune response in a subject in need thereof. Even further provided are the CDRs in an appropriate framework, variable regions, antibodies or antibody fragments thereof and/or compositions described herein for use in suppressing an immune response in a subject.

Determining whether a particular antibody or antibody fragment thereof can suppress an immune response can be assessed using endogenous GM-CSF or exogenous GM-CSF, for example from clinical samples like synovial fluid or GM-CSF produced in vitro by a human cancer, can be assessed using known in vitro immune assays including, but not limited to, inhibiting proinflammatory cytokine production; inhibiting phagocytosis or survival or morphology of neutrophils; inhibiting survival or morphology of eosinophils; detecting reduced HLA-DR, IL-1 beta, or proteases in monocytes or macrophages activation; inhibiting fusion of prefusion osteoclasts to activate them to resorb bone; inhibiting a cytotoxic T cell response including a CD8 T cell response by myeloid-derived suppressor cells; and any other assay that would be known to one of skill in the art to be useful in detecting inhibition of GM-CSF-mediated granulocyte, macrophage, dendritic cell or monocyte activation or GM-CSF-mediated innate or adaptive immune suppression.

The phrase “suppressing an immune response” as used herein refers to reducing the severity or duration of an immune response including, for example, reducing the activation of monocytes, macrophages, neutrophils, eosinophils and dendritic cells, and reduced inflammation. The phrase “suppressing an immune response” also includes reducing the action of GM-CSF increasing the number and activating myeloid-derived suppressor cells that inhibit the T cells that kill or inhibit cancer cells, including renal, colon, prostate, ovarian, melanoma, glioma, pancreatic and cervical cancers that produce GM-CSF. In an embodiment, suppressing an immune response refers to a 10%, 20%, 30% or more reduction compared to a subject that has not been treated.

In an embodiment, the methods and uses are for treating inflammation or an inflammatory disease in the subject in need thereof. In one embodiment, the inflammatory disease is arthritis, multiple sclerosis, asthma, chronic obstructive pulmonary disease (COPD), ankylosing spondylitis or psoriasis. In an embodiment, the inflammatory disease is arthritis. In a particular embodiment, the inflammatory disease is rheumatoid arthritis. In another embodiment, the methods and uses are for treating pain caused by cancer or osteoarthritis.

In another embodiment, the methods and uses are for treating cancer. In an embodiment, the treatment relieves myeloid-derived suppressor cells that inhibit immune response against the cancer. Cancers that can benefit from relief of myeloid-derived suppressor cells include renal, colon, prostate, ovarian, melanoma, glioma, pancreatic and cervical cancers that produce GM-CSF.

The disclosure further provides methods and uses of treating bone or cartilage loss, pain and/or juvenile myelomonocytic leukemia, all of which are adversely affected by GM-CSF. Accordingly, in one embodiment, there is provided a method of treating bone or cartilage loss, pain and/or juvenile myelomonocytic leukemia in a subject comprising administering an antibody or antibody fragment of the present disclosure, expression vectors expressing the isolated heavy and light chain of the present disclosure, a host cell of the present disclosure or a pharmaceutical composition of the present disclosure to the subject in need thereof. In another embodiment, there is provided a use of an antibody or antibody fragment of the present disclosure, expression vectors expressing the isolated heavy and light chain of the present disclosure, a host cell of the present disclosure or a pharmaceutical composition of the present disclosure for treating bone or cartilage loss, pain and/or juvenile myelomonocytic leukemia in a subject in need thereof. Further provided is a use of an antibody or antibody fragment of the present disclosure, expression vectors expressing the isolated heavy and light chain of the present disclosure, a host cell of the present disclosure or a pharmaceutical composition of the present disclosure in the preparation of a medicament for treating bone or cartilage loss, pain and/or juvenile myelomonocytic leukemia in a subject in need thereof. Even further provided is an antibody or antibody fragment of the present disclosure, expression vectors expressing the isolated heavy and light chain of the present disclosure, a host cell of the present disclosure or a pharmaceutical composition of the present disclosure for use in treating bone or cartilage loss, pain and/or juvenile myelomonocytic leukemia in a subject in need thereof.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilizing (i.e. not worsening) the state of disease, prevention of disease spread, delaying or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. Treatment methods comprise administering to a subject a therapeutically effective amount of an active agent and optionally consists of a single administration, or alternatively comprises a series of applications. The length of the treatment period depends on a variety of factors, such as the severity of the condition, the age of the patient, the concentration of active ingredient or agent, the activity of the compositions described herein, and/or a combination thereof. It will also be appreciated that the effective dosage of the agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

The term “subject” as used herein includes all members of the animal kingdom, including mammals, and suitably refers to humans.

The term “administering the CDRs in an appropriate framework, variable regions, antibodies or antibody fragments thereof” includes both the administration of protein as well as the administration of a nucleic acid sequence encoding the protein to an animal or to a cell in vitro or in vivo. The term “administering” also includes the administration of a cell that expresses the antibody or antibody fragment thereof.

The term “a cell” includes a single cell as well as a plurality or population of cells. Administering to a cell includes administering in vitro (or ex vivo) as well as in vivo.

The active agents or compositions of the present disclosure may be used alone or in combination with other known agents useful for suppressing the immune response, for example, for treating inflammation or cancer in a subject. When used in combination with other agents, it is an embodiment that the compositions or active agents of the present disclosure are administered contemporaneously with those agents. As used herein, “contemporaneous administration” of two substances to a subject means providing each of the two substances so that they are both biologically active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present disclosure that the composition or active agent of the present disclosure and the other agent(s) is administered to a subject in a non-contemporaneous fashion.

The dosage of compositions or active agents of the present disclosure can vary depending on many factors such as the pharmacodynamic properties of the composition, the mode of administration, the age, health and weight of the recipient, the nature and extent of the symptoms, the frequency of the treatment and the type of concurrent treatment, if any, and the clearance rate of the composition in the subject to be treated. One of skill in the art can determine the appropriate dosage based on, for example the above factors. Compositions of the present disclosure may be administered initially in a suitable dosage that may be adjusted as required, depending on the clinical response, and may be administered in a single daily dose or the total daily dose may be divided into two, three or four daily doses.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### Examples

The present inventor cloned recombinant human monoclonal antibody, F1, with methods detailed in patent application WO 2007/003041 A1. A gift of a sample of blood from a patient with IPAP was used. Peripheral blood mononuclear cells were prepared with Ficoll-Hypaque and frozen in liquid nitrogen. The cells were thawed in medium at 37° C. waterbath and put on ice on the further steps. The cells were further counted and the PBMC diluted with phosphate-buffered saline (PBS) with 2% fetal bovine serum (FBS). Human GM-CSF from Immunotools (Friesoythe, Germany) synthesized in E. coli had been previously labeled with Alexa-647, conjugated according to the manufacturer's instruction (Molecular Probes, Eugene, Oreg.). Human GM-CSF conjugated with Alexa-647 was added to the PBMC for 30 minutes on ice. The following antibodies were added with carefully titrated amounts of anti-CD3 conjugated with fluoroscein isothiocyanate (FITC) (purchased from BD Biosciences), polyclonal antibodies against anti-human IgM conjugated with FITC (purchased from Beckmann Coulter), monoclonal antibodies against anti-CD27 conjugated with phycoerythrin (purchased from BD Biosciences) and monoclonal antibodies against anti-CD19 conjugated with APC-H7 (purchased from BD Biosciences). After 30 minutes the cells were washed with PBS with 2% FBS. Annexin V-FITC was added in the buffer produced by the manufacturers for 10 minutes at room temperature and then the PBMC were washed with PBS with 2% FBS.

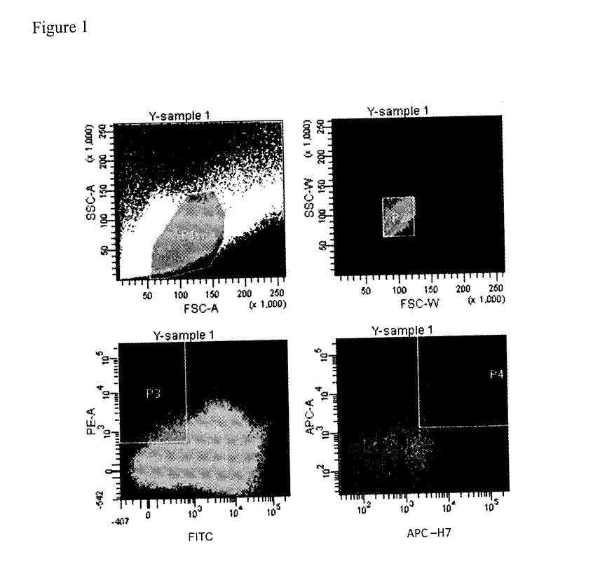

The enrichment of GM-CSF-specific individual memory B cells was done with the fluorescence-activated cell-sorter (FACS). The single memory B cells that were triple-stained with CD27, CD19 and GM-CSF Alexa-647 were placed by the FACS in each well of a 96-well microplate. First, single lymphocytes (P2, top right panel in FIG. 1) were gated on forward scatter and side-scatter. A gate, (P3, bottom left panel) was made by positive selection on anti-CD27 phycoerythrin and negative selection on anti-CD3-FITC (T lymphocytes), anti-IgM-FITC (IgM memory B cells) and Annexin V-FITC (apoptotic cells). Shown in P4, (bottom right panel) are the GM-CSF-binding CD19-positive and CD27-positive memory B cells on the upper-right quadrant.

The single B cells were expanded to clones of antibody-secreting cells with T cell helper cells and a mixture of cytokines (Wen, 1987). After a week incubation at 37° C., the supernatants of the wells were screened with an ELISA coated with GM-CSF.

The RNA from positive wells that the supernatants contained antibodies against GM-CSF screened in an ELISA, were purified and IgG primers used to make cDNA and then were amplified by PCR with primers according to current methods (Thomson, 2012). According to published methods, the variable regions of the H chain and L chain were sequenced and cloned into an expression vector with the respective IgG1 constant chain and the respective kappa or lambda constant chain (Thomson, 2012). They were then expressed in 293 HEK cells by transient expression and the IgG was purified with protein A (McLean, 2005).

The present inventor identified one of the human monoclonal antibodies against GM-CSF (F1) to have a potent ability to neutralize the bioactivity of GM-CSF. A standard assay was used to measure the ability to neutralize the bioactivity of GM-CSF on an erythroleukemia human leukemia cell-line (TF-1) that depends on the bioactivity of GM-CSF to survive or grow.

F1 was serially diluted, added to GM-CSF at 400 pg/mL, and pre-incubated at 37° C. for 1 hour. The F1 and GM-CSF mixtures were added to an equal volume of washed TF-1 cells, 1000 cells per well, yielding a final concentration of 200 pg/mL GM-CSF. After incubation at 37° C. for 4 days, WST-1 reagent (Roche cat #11 644 807 001) was added and incubation continued for 4 hrs. The absorbance values at wavelength 450 nm, with reference wavelength of 690 nm values subtracted, were determined using a plate reader. The absorbance values were directly proportional to the number of viable cells because the tetrazolium salts in the WST-1 reagent were cleaved to formazan by mitochondrial dehydrogenases in the cells. The blank values were subtracted from all wells on each plate. The % inhibition of the mAbs was calculated from the OD values using the wells with only GM-CSF and no GM-CSF or F1 as the reference. The IC50 of F1 was 0.7-0.8 ng/mL reproducibly. Other experiments showed that an irrelevant human monoclonal IgG1 antibody, KE5, a human monoclonal antibody that neutralized human cytomegalovirus had no inhibition on TF-1 growth in GM-CSF. To control that the F1 did not inhibit the growth of TF-1 cells, the GM-CSF was replaced by a final concentration of 1% (v/v) gibbon IL-3-conditioned medium, which TF-1 grows as well as GM-CSF. F1 did not in repeated experiments inhibit TF-1 growth and survival supported by IL-3.

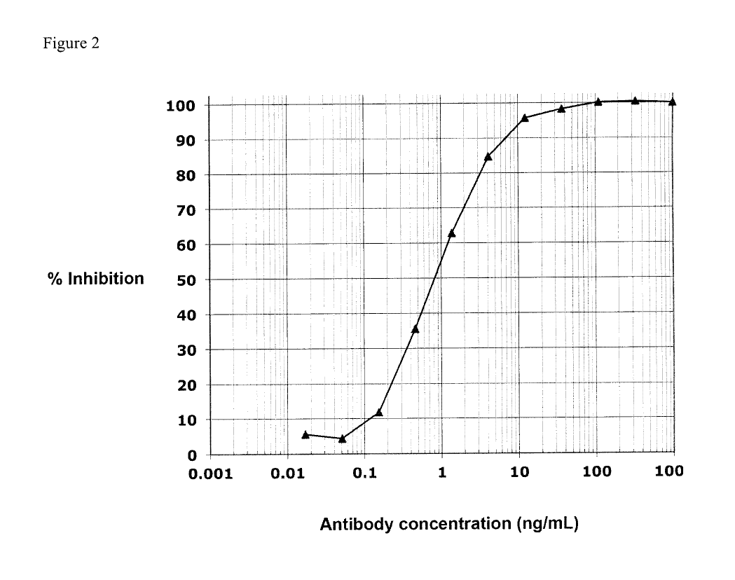

The potent F1 monoclonal antibody had an IC50 of less than a 1 ng/mL in the TF-1 assay (FIG. 2).

In contrast, Li et al (Li, 2006) reported an IC50 about 1,000-10,000 fold higher than the F1 antibody in a TF-1 assay. Li et al reported results of two monoclonal antibodies, one was an IC50 of about 1 ug/mL and the other was with an IC50 of about 10 ug/mL. In other words, Li et al. antibodies were 1,000-10,000 fold less potent than the presently disclosed F1 antibody. Takada et al WO/2009/064399 generated four human monoclonal antibodies against human GM-CSF that were much less potent than F1. EV003 was the most potent as a single monoclonal antibody although it was only able to neutralize at a high 2 ug/ml concentration. Furthermore, even at a concentration of 2 ug/ml, 20% of the TF-1 were still alive. EV1018 and EV1019 had abnormal dose-response curves in the TF-1 assay that did not drop with higher concentrations of monoclonal antibody below ˜50% proliferation with TF-1 stimulated with yeast GM-CSF, and below ˜40% with TF-1 stimulated with E. coli GM-CSF. However the dose-response curves dropped to zero TF-1 proliferation, when two monoclonal antibodies against GM-CSF that bound to different epitopes were added.

In addition, Takada et al held cultured the EBV-transformed B cells for 12-16 weeks and Activation Induced cytidine Deaminase (AID), the enzyme that physiologically introduces somatic mutations into antibodies, which is highly expressed in EBV-transformed B cells. Thus, AID would have somatically mutated the antibodies randomly for 12-16 weeks (Heath, 2012) before the antibodies were cloned and sequenced. In contrast to the germinal centre in vivo, there was no antigen in vitro and there were no methods of selection to increase affinity of the antibody or selection methods to maintain the thermodynamic stability of the antibody (Wang, Sen, 2013). The monoclonal antibodies developed by the method of Takada et al have the potential for thermodynamic instability and could develop aggregates and immunogenicity.

In contrast, the increased potency of the F1 antibody is beneficial for clinical use as a more potent monoclonal antibody. It is easy for the patient to inject themselves or the patient can go longer intervals between injections. It is also less expensive to manufacture the antibody, as it costs less to treat a patient effectively with smaller doses. The high potency against the bioactivity of GM-CSF depends on both the affinity and the site at which the monoclonal antibodies bind to the GM-CSF.

To further test the ability of the F1 monoclonal antibody to neutralize bioactivity of GM-CSF another test was used, normal human neutrophils stimulated with GM-CSF. High concentrations of GM-CSF (10 ng/mL) are known to induce more levels of a surface marker called CD11b. Titrations of F1 or growth medium were pre-incubated at 37° C. for 60 min with GM-CSF. Triplicate 100 μL samples of heparinized human whole blood were combined with the pre-incubated F1 and GM-CSF mixtures and incubation was continued at 37° C. for an additional 30 min. The final concentration of GM-CSF in the blood was 10 ng/mL and that of the F1 varied from 2-300 ng/mL. Red blood cells were lysed in 6 mL of H₂O for 30 sec, and then 2 mL of PBS with 2.5% NaCl and 6 mL of KRG buffer were added. Cells were stained with anti-human CD11b-PE Ab on ice for 30 min. The cells were washed and FACS was performed to evaluate CD11b levels on the neutrophils, which were gated on high side-scatter. The changes in CD11 b levels on neutrophils were calculated as the mean fluorescence intensity of CD11b on neutrophils stimulated by GM-CSF minus that of CD11b on the mixture of GM-CSF with mAb, divided by the mean fluorescence intensity of GM-CSF stimulated neutrophils, multiplied by 100.

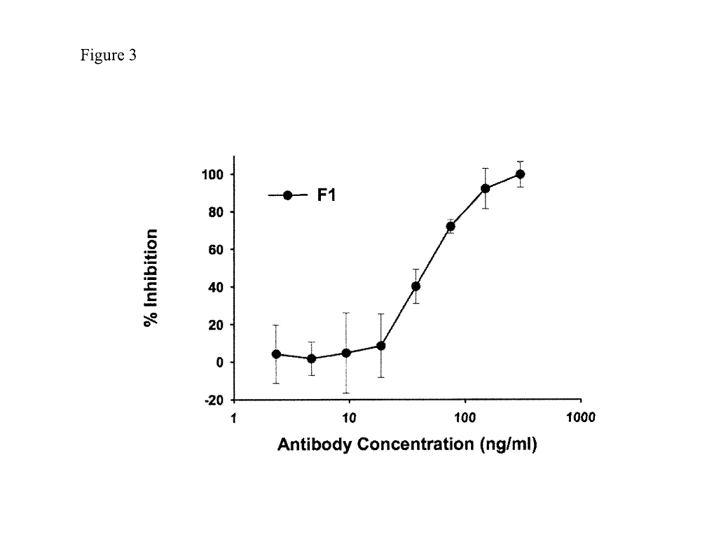

As can be seen in FIG. 3, F1 neutralized the bioactivity of GM-CSF on primary human neutrophils with high concentrations of GM-CSF (10 ng/mL).

Surface plasmon resonance was used to measure the kinetics and on rate and off rate and the affinity of F1 (see Table 1). The SPR experiments were performed at 25° C. on a Biacore 3000 (GE Healthcare, Piscataway, N.J.) using a CM5 sensor chip and HBS-P (10 mM HEPES, pH 7.4, 150 mM NaCl, 0.01% P-20) or HBS-EP (10 mM HEPES, pH 7.4, 150 mM NaCl, 3 mM EDTA, 0.01% P-20). Anti-human IgG (Fc) (GE Healthcare) or anti-human IgG (Southern Biotech, cat. #2040-01) was immobilized similarly on the experimental and reference flow cells using standard amine coupling. For affinity measurements, individual anti-GM-CSF mAbs were injected and captured on flow cells 2 or 4 leaving flow cells 1 and 3 as reference. GM-CSF made in-house or purchased (Genscript, Cat. #Z00349), was injected over the mAb-coated cell and reference cell at the following concentrations: 100, 50, 25, 12.5, 6.25, 3.12, 1.5 and 0.8 nM. The flow rate was 30 μL/min and triplicate blank injections and replicate injections of the 12.5 nM GM-CSF were included to account for any drift and to ensure assay reproducibility. Because the interaction between many of the mAbs with the rhGM-CSF was high-affinity, a long dissociation phase (upwards of 3000 sec) was used to accurately determine the dissociation rate constant (kd) for those mAbs where it was necessary (Drake, 2004). To regenerate the capture surface, two 30 sec pulses of a 2:1 mixture of glycine (pH 2) to 3 M MgCI was utilized. All data were double reference subtracted in that they were subtracted both from the reference cell and from a blank injection. Data were fit to a 1:1 model with (for those with very fast on-rates) or without a mass transport term using the Biacore 3000 evaluation software. Table 1 shows the kinetics and on-rate and off-rate and the affinity data for three human monoclonal antibodies against GM-CSF from IPAP patients, F1, C3 and C5. The K_{D} of F1 was 1.2×10⁻¹⁰ M, which demonstrates a very high affinity antibody. C3 had a higher affinity than F1 and C3 had a higher IC50 than F1. The present inventor hypothesized that F1 might bind to a separate epitope than the other human monoclonal antibodies against GM-CSF so the epitope that F1 bound on GM-CSF was investigated.

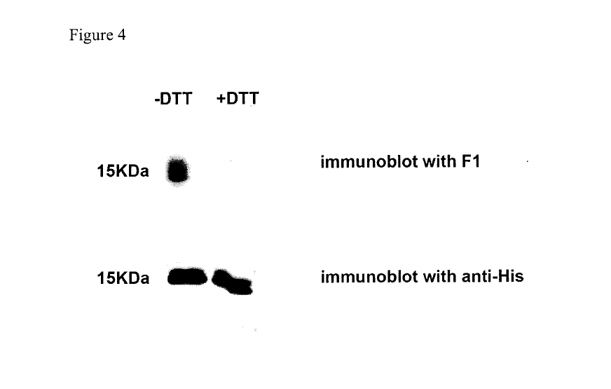

F1 did not bind to a linear peptide epitope but a conformational epitope (FIG. 4). Denatured recombinant GM-CSF containing an 8×-His tag produced from E. coli was boiled and reduced with dithiothreitol (DTT) in the presence of SDS. Human GM-CSF has two disulfide bonds (Diederichs, 1991). It was run in multiple lanes on SDS-PAGE and transferred to a nitrocellulose membrane. The individual lanes were cut and immunoblotted with human mAbs targeting GM-CSF and a mouse anti-His monoclonal antibody. Note that F1 did not bind to denatured, reduced GM-CSF but that the anti-His monoclonal antibody bound to the His tag on denatured reduced GM-CSF.

The epitope bound by F1 was then investigated by surface plasmon resonance (SPR) and, for comparison, other human monoclonal antibodies generated from IPAP patients against GM-CSF that had higher IC50 in the TF-1 assay namely A1, A2, B1, C2, C3, C5, E1, E2, E3, E5, E6 and E7. For epitope mapping by surface plasmon resonance (SPR), the individual anti-GM-CSF monoclonal antibodies did not have to be labeled. The SPR experiments were performed at 25° C. on a Biacore 3000 using a CM5 sensor chip and anti-human IgG was immobilized similarly on the experimental and reference flow cells using standard amine coupling. Each individual human monoclonal antibody against GM-CSF was injected and captured on anti-human IgG bound to the chip. Polyclonal hIgG was injected to block any free anti-human IgG binding sites on the chip surface from the immobilized anti-IgG. The GM-CSF was then injected and captured by the first monoclonal antibody. This was followed by an injection of a second anti-GM-CSF monoclonal antibody, followed by a third anti-GM-CSF monoclonal antibody. If binding was observed for the second mAb but not the third mAb, it was not assumed that the lack of binding of the third mAb was due to the first captured mAb because it could be due to the second anti-GM-CSF monoclonal antibody. The majority of the epitope mapping was performed such that monoclonal antibodies were tested both as being captured on the chip surface and being floated as analyte to confirm the mapping interactions.

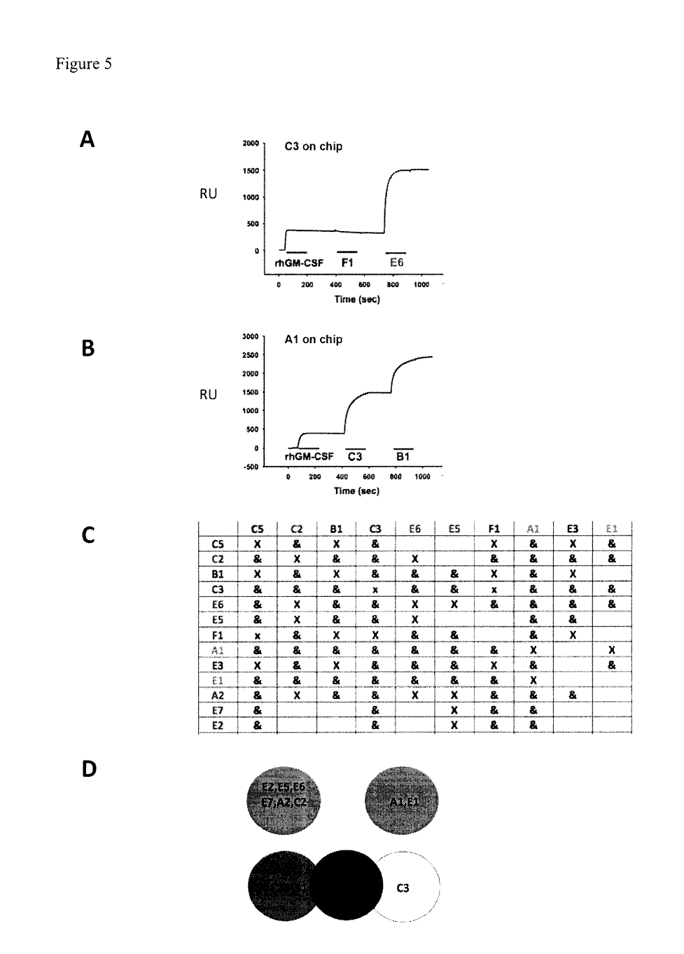

Three different human monoclonal antibodies against GM-CSF are shown in FIG. 5A. A monoclonal antibody against GM-CSF, C3 was attached to the chip and C3 captured GM-CSF flowing over the chip. Then F1 was flowed over the chip but it did not bind to the GM-CSF captured by C3. That means that F1 and C3 cannot bind GM-CSF simultaneously and their epitopes overlap. A third monoclonal antibody, E6, was injected and it bound to the GM-CSF captured by C3. So A1 and E6 could simultaneously bind a single molecule of GM-CSF and they bound to non-overlapping epitopes. Shown in FIG. 5B is another monoclonal antibody, A1, attached to the chip. The GM-CSF was flowed over the chip and captured by A1. Then C3 was flowed over the chip and bound to GM-CSF. That means that A1 and C3 can simultaneously bind to a single molecule of GM-CSF. The GM-CSF bound by A1 and C3 is further bound by a third monoclonal antibody, B1. FIG. 5C shows a summary of whether a monoclonal antibody can bind simultaneously with another monoclonal antibody to the same molecule of GM-CSF or not. FIG. 5D shows a diagram constructed from FIG. 5C that demonstrates that F1 binds to an epitope that spans two non-overlapping epitopes of GM-CSF, one is the epitope bound by C3 and the second includes the epitope bound by C5.

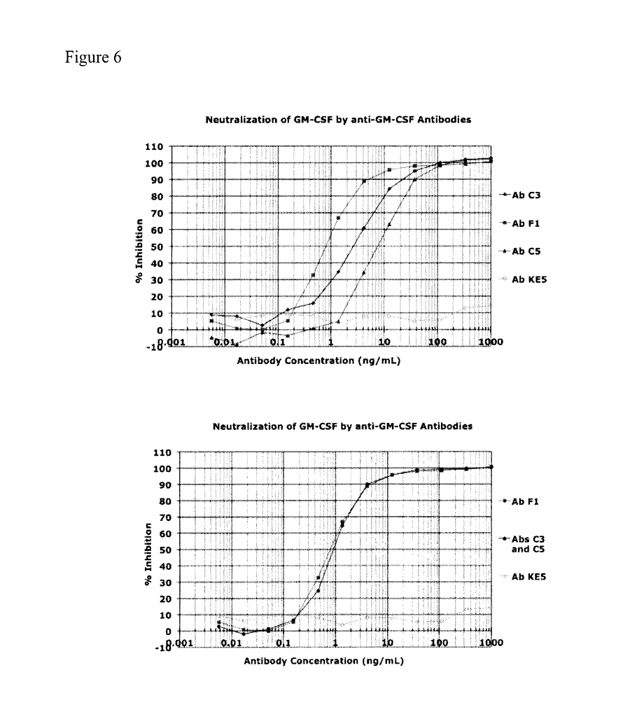

C3 is the highest affinity antibody against GM-CSF, and C5 a high affinity antibody (Table 1). Done in the same experiment (FIG. 6) is a titration of F1, C3 and C5 and an equal ratio of a mixture of C3 and C5 with the total IgG on the X axis. An equal mixture of two monoclonal antibodies, C3 and C5, shows synergy in neutralizing the bioactivity of GM-CSF in the TF-1 assay (FIG. 6). Two panels are shown with C3 and C5 singly titrated in the top panel and an equal mixture of C3 and C5 shown in the bottom. For clarity, the titration of F1 and KE5 is drawn in both the top and bottom panels. F1 has the same IC50 as the synergistic mixture of C3 and C5 (FIG. 6). (KE5, an IgG1 human monoclonal antibody that neutralized human cytomegalovirus, was used as a negative control).

C3 and C5 bind simultaneously to GM-CSF and correspondingly bind to non-overlapping epitopes (FIG. 5C, 5D). However, C3 or C5 neutralize the bioactivity of GM-CSF (FIG. 6) by sterically hindering different sites of the GM-CSF surface area with their interaction with sites on the GM-CSF receptor. C3 or C5 neutralize the bioactivity of GM-CSF with less potency than F1 and have higher IC50's (Table 1). F1 had lower affinity for GM-CSF than C3 but was more potent (Table 1). The F1 epitope on GM-CSF overlaps the epitopes bound by C3 and C5 (FIGS. 5C and 5D). F1 when binds to GM-CSF, it very efficiently sterically hinders the binding of GM-CSF to its receptor, perhaps sterically hindering the different sites GM-CSF interacting with the GM-CSF receptor. As C3 and C5 bind to a single molecule of GM-CSF at different sites on GM-CSF (FIG. 5C) and they both neutralize the bioactivity of GM-CSF by sterically hindering two sites on GM-CSF that interact with different sites on the GM-CSF receptor signaling complex, the inventor hypothesized that C3 and C5 should synergize to neutralize bioactivity of GM-CSF. Furthermore, as IgG1 is bivalent, two molecules of GM-CSF would form a stable, high-avidity tetrameric complex with C3 and C5, as demonstrated by Moyle et al (Moyle, 1983). As was shown in FIG. 6, an equal mixture of C3 and C5 had the same potency at neutralizing the GM-CSF bioactivity as F1. With F1 spanning the non-overlapping epitopes of C3 and C5 (FIGS. 5C and 5D), without wishing to be bound by theory, this may explain how targeting the epitope of F1 is very efficient at neutralizing the GM-CSF bioactivity inhibiting two different sites on GM-CSF interacting with the GM-CSF receptor.

### DISCUSSION

Because the F1 antibody is a copy of an antibody that was generated naturally in a human, and has gone through the quality control systems of the immune system, it is likely to be less immunogenic or aggregate than current artificial monoclonal antibodies on the market that have been generated by genetic engineering. The present inventor produced artificial cDNA with IgG primers with SuperScript III Reverse Transcriptase (Invitrogen) followed by PCR-amplification using Platinum pfx and Platinum Taq DNA Polymerase (Invitrogen) with standard primers. The present inventor then cloned both the cDNA's of the variable regions of the H and L chains and expressed them transiently in vectors with the constant region of the H chain and the kappa chain.

Antibodies against a monoclonal antibody cause reactions at the injection site, decrease patient acceptance and compliance and problems with efficacy may cause deterioration of the disease. The issue of the immunogenicity of the monoclonal antibody is especially important for chronic conditions when the treatment will have to be continued for decades.

The other problem for use in humans is that an unnatural, inauthentic pairing of H and L chains may make the antibody prone to aggregation and be immunogenic to humans (Rosenberg, 2006). The artificial monoclonal antibody adalimumab that was generated by genetic engineering from human H and L chain genes and has unnatural pairing of H and L chains, generates antibodies in 28% of patients (Bartelds, 2011) and increased clearance of the antibody and impaired treatment responses.

Moreover, human monoclonal antibodies generated through genetic engineering that were not generated naturally in humans, have not undergone the quality control of the human immune system which rejects ˜50% of human antibodies as autoreactive (Wardemann, 2003). Thus, artificial, engineered “human” monoclonal antibodies can show auto-reactivity (Wu, 2007). The presently disclosed human mAbs have passed this natural quality control system and have undergone affinity-maturation, which includes maintaining thermodynamic stability (Wang, Sen 2013). Using the authentic paired H and L variable sequences along with either natural or non-natural constant regions that encode such a potent antibody, it is possible to produce these quality controlled antibodies through various techniques, including recombinant technology. Wang et al (Wang, Sen 2013) found that monoclonal antibodies that had gone through affinity maturation were optimized for protein stability and expression.

While the present disclosure has been described with reference to what are presently considered to be the examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Kinetics, dissociation constant, and IC50 for F1, C3 and C5

| | Ka × 10⁵ | kd × 10⁻⁴ | K_{D} × 10⁻¹⁰ | IC50 |
|---|---|---|---|---|
| Antibody | (1/Ms) | (1/s) | (M) | (ng/mL) |
| F1 | 8.3 | 1.0 | 1.2 | 0.8 |
| C3 | 12.0 | 0.44 | 0.37 | 2.7 |
| C5 | 12.0 | 3.7 | 3.0 | 7.5 |

TABLE 2
Polypeptide CDRH
SEQ ID NO: 1 CDRH1 DSAIRKYY
SEQ ID NO: 2 CDRH2 IYASGSS
SEQ ID NO: 3 CDRH3 AAITGTTDL
SEQ ID NO: 17 CDRH2 IYASGSS F
(CDR2 plus first amino acid of FR3)
SEQ ID NO: 19 atctatgccagtgggagttctttt
(encoding CDR2 + Phenylalanine)
Polynucleotide CDRH
SEQ ID NO: 4 CDRH1 gatagtgccatcaggaaatactat
SEQ ID NO: 5 CDRH2 atctatgccagtgggagttct
SEQ ID NO: 6 CDRH3 gcggccataactggaacgactgatctc
Polypeptide H chain
SEQ ID NO: 13
QVQLQESGPGVVKPSETLSLTCSVSDSAIRKYYWSWIRQPPGQGLEYIGY
IYASGSSFYNPSFKSRVSMSVDATNNQFYLKLTSVTAADTAVYYCAAITG
TTDL
SEQ ID NO: 21:
QVQLQESGPGVVKPSETLSLTCSVSDSAIRKYYWSWIRQPPGQGLEYIGY
IYASGSSFYNPSFKSRVSMSVDATNNQFYLKLTSVTAADTAVYYCAAITG
TTDLWGRGTLVTVSS
Polynucleotide H chain
SEQ ID NO: 15
CAGGTGCAGCTGCAGGAGTCGGGCCCAGGAGTGGTGAAGCCTTCGGAG
ACCCTGTCCCTCACCTGCAGTGTCTCTGATAGTGCCATCAGGAAATACTA
TTGGAGTTGGATCCGGCAGCCCCCAGGACAGGGACTGGAGTATATTGG
ATATATCTATGCCAGTGGGAGTTCTTTTTATAATCCCTCCTTCAAGAGTC
GAGTCAGCATGTCGGTAGACGCGACCAACAATCAGTTCTACCTGAAGTTG
ACTTCTGTCACCGCCGCGGACACGGCCGTATATTACTGTGCGGCCATAA
CTGGAACGACTGATCTC
SEQ ID NO: 23
CAGGTGCAGCTGCAGGAGTCGGGCCCAGGAGTGGTGAAGCCTTCGGAG
ACCCTGTCCCTCACCTGCAGTGTCTCTGATAGTGCCATCAGGAAATACTA
TTGGAGTTGGATCCGGCAGCCCCCAGGACAGGGACTGGAGTATATTGG
ATATATCTATGCCAGTGGGAGTTCTTTTTATAATCCCTCCTTCAAGAGTC
GAGTCAGCATGTCGGTAGACGCGACCAACAATCAGTTCTACCTGAAGTTG
ACTTCTGTCACCGCCGCGGACACGGCCGTATATTACTGTGCGGCCATAA
CTGGAACGACTGATCTCTGGGGCCGTGGCACCCTGGTCACTGTCTCCTC
AG
Polypeptide CDRL
SEQ ID NO: 7 CDRL1 QGINRR
SEQ ID NO: 8 CDRL2 AVS
SEQ ID NO: 9 CDRL3 LQSNNYPLT
SEQ ID NO: 18 CDRL2 AVS T
(CDR2 plus first amino acid of FR3)
SEQ ID NO: 20 gctgtgtccact
(encoding CDRL2 + Threonine)
Polynucleotides CDRL
SEQ ID NO: 10 CDRL1 cagggaattaacaggagg
SEQ ID NO: 11 CDRL2 gctgtgtcc
SEQ ID NO: 12 CDRL3 ctacagtctaacaactatcccctcact
Polypeptide L chain
SEQ ID NO: 14
DIQMTQSPSSVSASVGDRVTITCRASQGINRRLAWYQQKPGKAPKRLIYA
VSTLQSGVPSRFNGSGSGTDFTLTVNNVQPDDLAMYFCLQSNNYPLT
SEQ ID NO: 22
DIQMTQSPSSVSASVGDRVTITCRASQGINRRLAWYQQKPGKAPKRLIYA
VSTLQSGVPSRFNGSGSGTDFTLTVNNVQPDDLAMYFCLQSNNYPLTFGG
GTKVEIK
Polynucleotides L chain
SEQ ID NO: 16
GACATCCAGATGACCCAGTCTCCATCTTCTGTGTCTGCTTCTGTAGGAGA
CAGGGTCACCATCACTTGCCGGGCGAGTCAGGGAATTAACAGGAGGTTA
GCCTGGTATCAGCAGAAACCAGGGAAAGCCCCTAAACGCCTGATCTATG
CTGTGTCCACTTTGCAAAGTGGAGTCCCATCCCGATTCAACGGCAGTGG
ATCTGGGACAGATTTCACTCTCACTGTCAATAACGTGCAGCCTGATGATC
TTGCAATGTATTTTTGTCTACAGTCTAACAACTATCCCCTCACT
SEQ ID NO: 24
GACATCCAGATGACCCAGTCTCCATCTTCTGTGTCTGCTTCTGTAGGAGA
CAGGGTCACCATCACTTGCCGGGCGAGTCAGGGAATTAACAGGAGGTTA
GCCTGGTATCAGCAGAAACCAGGGAAAGCCCCTAAACGCCTGATCTATG
CTGTGTCCACTTTGCAAAGTGGAGTCCCATCCCGATTCAACGGCAGTGG
ATCTGGGACAGATTTCACTCTCACTGTCAATAACGTGCAGCCTGATGATC
TTGCAATGTATTTTTGTCTACAGTCTAACAACTATCCCCTCACTTTCGGC
GGTGGGACCAAGGTGGAGATCAAAC

### REFERENCES

1. Babcook, J. S., K. B. Leslie, O. A. Olsen, R. A. Salmon, and J. W. Schrader. 1996. A novel strategy for generating monoclonal antibodies from single, isolated lymphocytes producing antibodies of defined specificities. Proceedings of the National Academy of Sciences of the United States of America 93:7843-7848.
2. Bartelds, G. M., C. L. Krieckaert, M. T. Nurmohamed, P. A. van Schouwenburg, W. F. Lems, J. W. Twisk, B. A. Dijkmans, L. Aarden, and G. J. Wolbink. 2011. Development of antidrug antibodies against adalimumab and association with disease activity and treatment failure during long-term follow-up. Jama 305:1460-1468.
3. Bayne, L. J., G. L. Beatty, N. Jhala, C. E. Clark, A. D. Rhim, B. Z. Stanger, and R. H. Vonderheide. 2012. Tumor-derived granulocyte-macrophage colony-stimulating factor regulates myeloid inflammation and T cell immunity in pancreatic cancer. Cancer Cell 21:822-835.
4. Brochet, X., M. P. Lefranc, and V. Giudicelli. 2008. IMGT/V-QUEST: the highly customized and integrated system for IG and TR standardized V-J and V-D-J sequence analysis. Nucleic Acids Res 36:W503-508.
5. Bronte, V., D. B. Chappell, E. Apolloni, A. Cabrelle, M. Wang, P. Hwu, and N. P. Restifo. 1999. Unopposed production of granulocyte-macrophage colony-stimulating factor by tumors' inhibits CD8+T cell responses by dysregulating antigen-presenting cell maturation. J Immunol 162:5728-5737.
6. Burmester, G. R., E. Feist, M. A. Sleeman, B. Wang, B. White, and F. Magrini. 2011. Mavrilimumab, a human monoclonal antibody targeting GM-CSF receptor-alpha, in subjects with rheumatoid arthritis: a randomised, double-blind, placebo-controlled, phase I, first-in-human study. Ann Rheum Dis 70:1542-1549.
7. Codarri, L., G. Gyulveszi, V. Tosevski, L. Hesske, A. Fontana, L. Magnenat, T. Suter, and B. Becher. 2011. RORgammat drives production of the cytokine GM-CSF in helper T cells, which is essential for the effector phase of autoimmune neuroinflammation. Nat Immunol 12:560-567.
8. Cook, A. D., E. L. Braine, I. K. Campbell, M. J. Rich, and J. A. Hamilton. 2001. Blockade of collagen-induced arthritis post-onset by antibody to granulocyte-macrophage colony-stimulating factor (GM-CSF): requirement for GM-CSF in the effector phase of disease. Arthritis Res 3:293-298.
9. Cook, A. D., J. Pobjoy, S. Sarros, S. Steidl, M. Durr, D. C. Lacey, and J. A. Hamilton. 2013. Granulocyte-macrophage colony-stimulating factor is a key mediator in inflammatory and arthritic pain. Ann Rheum Dis 72:265-270.
10. Diederichs, K., T. Boone, and P. A. Karplus. 1991. Novel fold and putative receptor binding site of granulocyte-macrophage colony-stimulating factor. Science 254:1779-1782.
11. Drake, A. W., D. G. Myszka, and S. L. Klakamp. 2004. Characterizing high-affinity antigen/antibody complexes by kinetic- and equilibrium-based methods. Anal Biochem 328:35-43.
12. El-Behi, M., B. Ciric, H. Dal, Y. Yan, M. Cullimore, F. Safavi, G. X.

Zhang, B. N. Dittel, and A. Rostami. The encephalitogenicity of T(H)17 cells is dependent on IL-1- and IL-23-induced production of the cytokine GM-CSF. Nat Immunol 12:568-575.
13. Epeldegui, M., Y. P. Hung, A. McQuay, R. F. Ambinder, and O. Martinez-Maza. 2007. Infection of human B cells with Epstein-Barr virus results in the expression of somatic hypermutation-inducing molecules and in the accrual of oncogene mutations. Mol Immunol 44:934-942.
14. Frei, K., D. Piani, U. V. Malipiero, E. Van Meir, N. de Tribolet, and A. Fontana. 1992. Granulocyte-macrophage colony-stimulating factor (GM-CSF) production by glioblastoma cells. Despite the presence of inducing signals GM-CSF is not expressed in vivo. J Immunol 148:3140-3146.
15. Gigliotti, F., L. Smith, and R. A. Inset. 1984. Reproducible production of protective human monoclonal antibodies by fusion of peripheral blood lymphocytes with a mouse myeloma cell line. J Infect Dis 149:43-47.
16. Gil, Y., S. Levy-Nabot, M. Steinitz, and R. Laskov. 2007. Somatic mutations and activation-induced cytidine deaminase (AID) expression in established rheumatoid factor-producing lymphoblastoid cell line. Mol Immunol 44:494-505.
17. Giudicelli, V., X. Brochet, and M. P. Lefranc. 2011. IMGT/V-QUEST: IMGT standardized analysis of the immunoglobulin (IG) and T cell receptor (TR) nucleotide sequences. Cold Spring Harbor protocols 2011:695-715.
18. Gualtieri, R. J., P. D. Emanuel, K. S. Zuckerman, G. Martin, S. C. Clark, R. K. Shadduck, R. A. Dracker, J. Akabutu, R. Nitschke, M. L. Hetherington, and et al. 1989. Granulocyte-macrophage colony-stimulating factor is an endogenous regulator of cell proliferation in juvenile chronic myelogenous leukemia. Blood 74:2360-2367.
19. Hamilton, J. A. 2008. Colony-stimulating factors in inflammation and autoimmunity. Nat Rev Immunol 8:533-544.
20. Hamilton, J. A., and A. Achuthan. 2013. Colony stimulating factors and myeloid cell biology in health and disease. Trends Immunol 34:81-89.
21. Hamilton, J. A., E. R. Stanley, A. W. Burgess, and R. K. Shadduck. 1980. Stimulation of macrophage plasminogen activator activity by colony-stimulating factors. J Cell Physiol 103:435-445.
22. Handman, E., and A. W. Burgess. 1979. Stimulation by granulocyte-macrophage colony-stimulating factor of Leishmania tropica killing by macrophages. J Immunol 122:1134-1137.
23. Hansen, G., T. R. Hercus, B. J. McClure, F. C. Stomski, M. Dottore, J. Powell, H. Ramshaw, J. M. Woodcock, Y. Xu, M. Guthridge, W. J. McKinstry, A. F. Lopez, and M. W. Parker. 2008. The structure of the GM-CSF receptor complex reveals a distinct mode of cytokine receptor activation. Cell 134:496-507.
24. He, B., N. Raab-Traub, P. Casali, and A. Cerutti. 2003. EBV-encoded latent membrane protein 1 cooperates with BAFF/BLyS and APRIL to induce T cell-independent Ig heavy chain class switching. J Immunol 171:5215-5224.
25. Heath, E., N. Begue-Pastor, S. Chaganti, D. Croom-Carter, C. Shannon-Lowe, D. Kube, R. Feederle, H. J. Delecluse, A. B. Rickinson, and A. I. Bell. 2012. Epstein-Barr virus infection of naive B cells in vitro frequently selects clones with mutated immunoglobulin genotypes: implications for virus biology. PLoS Pathog 8:e1002697.
26. Hercus, T. R., D. Thomas, M. A. Guthridge, P. G. Ekert, J. King-Scott, M. W. Parker, and A. F. Lopez. 2009. The granulocyte-macrophage colony-stimulating factor receptor: linking its structure to cell signaling and its role in disease. Blood 114:1289-1298.
27. Iversen, P. O., I. D. Lewis, S. Turczynowicz, H. Hasle, C. Niemeyer, K. Schmiegelow, S. Bastiras, A. Biondi, T. P. Hughes, and A. F. Lopez. 1997. Inhibition of granulocyte-macrophage colony-stimulating factor prevents dissemination and induces remission of juvenile myelomonocytic leukemia in engrafted immunodeficient mice. Blood 90:4910-4917.
28. Kitamura, T., N. Tanaka, J. Watanabe, Uchida, S. Kanegasaki, Y. Yamada, and K. Nakata. 1999. Idiopathic pulmonary alveolar proteinosis as an autoimmune disease with neutralizing antibody against granulocyte/macrophage colony-stimulating factor. J Exp Med 190:875-880.
29. Koike, K., and K. Matsuda. 2008. Recent advances in the pathogenesis and management of juvenile myelomonocytic leukaemia. British Journal of Haematology 141:567-575%*-© 2008 The Authors. Journal compilation-© 2008 Blackwell Publishing Ltd % U http://onlinelibrary.wiley.com/doi/2010.1111/j.1365-2141.2008.07104.x/abstract.
30. Krause, J. C., T. Tsibane, T. M. Tumpey, C. J. Huffman, R. Albrecht, D. L. Blum, I. Ramos, A. Fernandez-Sesma, K. M. Edwards, A. Garcia-Sastre, C. F. Basler, and J. E. Crowe, Jr. 2012. Human monoclonal antibodies to pandemic 1957 H2N2 and pandemic 1968 H3N2 influenza viruses. J Virol 86:6334-6340.
31. Lee, M. S., H. S. Kim, J.-T. Yeon, S.-W. Choi, C. H. Chun, H. B. Kwak, and J. Oh. 2009. GM-CSF regulates fusion of mononuclear osteoclasts into bone-resorbing osteoclasts by activating the Ras/ERK pathway. Journal of Immunology (Baltimore, Md.: 1950) 183:3390-3399% U http://www.ncbi.nlm.nih.qov/pubmed/19641137.
32. Li, J., T. Sal, M. Berger, Q. Chao, D. Davidson, G. Deshmukh, B. Drozdowski, W. Ebel, S. Harley, M. Henry, S. Jacob, B. Kline, E. Lazo, F. Rotella, E. Routhier, K. Rudolph, J. Sage, P. Simon, J. Yao, Y. Zhou, M. Kavuru, T. Bonfield, M. J. Thomassen, P. M. Sass, N. C. Nicolaides, and L. Grasso. 2006. Human antibodies for immunotherapy development generated via a human B cell hybridoma technology. Proc Natl Acad Sci USA 103:3557-3562.
33. Lindau, D., P. Gielen, M. Kroesen, P. Wesseling, and G. J. Adema. 2013. The immunosuppressive tumour network: myeloid-derived suppressor cells, regulatory T cells and natural killer T cells. Immunology 138:105-115.
34. Lopez, A. F., D. J. Williamson, J. R. Gamble, C. G. Begley, J. M. Harlan, S. J. Klebanoff, A. Waltersdorph, G. Wong, S. C. Clark, and M. A. Vadas. 1986. Recombinant human granulocyte-macrophage colony-stimulating factor stimulates in vitro mature human neutrophil and eosinophil function, surface receptor expression, and survival. J Clin Invest 78:1220-1228.
35. McLean, G. R., 0. A. Olsen, I. N. Watt, P. Rathanaswami, K. B. Leslie, J. S. Babcook, and J. W. Schrader. 2005. Recognition of human cytomegalovirus by human primary immunoglobulins identifies an innate foundation to an adaptive immune response. J Immunol 174:4768-4778.
36. McQualter, J. L., R. Darwiche, C. Ewing, M. Onuki, T. W. Kay, J. A. Hamilton, H. H. Reid, and C. C. Bernard. 2001. Granulocyte macrophage colony-stimulating factor: a new putative therapeutic target in multiple sclerosis. J Exp Med 194:873-882.
37. Metcalf, D. 2008. Hematopoietic cytokines. Blood 111:485-491.
38. Moyle, W. R., D. M. Anderson, and P. H. Ehrlich. 1983. A circular antibody-antigen complex is responsible for increased affinity shown by mixtures of monoclonal antibodies to human chorionic gonadotropin. Journal of Immunology (Baltimore, Md.: 1950) 131:1900-1905% U http://www.ncbi.nlm.nih.qov/pubmed/6619544.
39. Okada, Y., C. Terao, K. Ikari, Y. Kochi, K. Ohmura, A. Suzuki, T. Kawaguchi, E. A. Stahl, F. A. Kurreeman, N. Nishida, H. Ohmiya, K. Myouzen, M. Takahashi, T. Sawada, Y. Nishioka, M. Yukioka, T. Matsubara, S. Wakitani, R. Teshima, S. Tohma, K. Takasugi, K. Shimada, A. Murasawa, S. Honjo, K. Matsuo, H. Tanaka, K. Tajima, T. Suzuki, T. Iwamoto, Y. Kawamura, H. Tanii, Y. Okazaki, T. Sasaki, P. K. Gregersen, L. Padyukov, J. Worthington, K. A. Siminovitch, M. Lathrop, A. Taniguchi, A. Takahashi, K. Tokunaga, M. Kubo, Y. Nakamura, N. Kamatani, T. Mimori, R. M. Plenge, H. Yamanaka, S. Momohara, R. Yamada, F. Matsuda, and K. Yamamoto. 2012. Meta-analysis identifies nine new loci associated with rheumatoid arthritis in the Japanese population. Nat Genet 44:511-516.
40. Plater-Zyberk, C., L. A. Joosten, M. M. Helsen, J. Hepp, P. A. Baeuerle, and W. B. van den Berg. 2007. GM-CSF neutralisation suppresses inflammation and protects cartilage in acute streptococcal cell wall arthritis of mice. Ann Rheum Dis 66:452-457.
41. Pylayeva-Gupta, Y., K. E. Lee, C. H. Hajdu, G. Miller, and D. Bar-Sagi. 2012. Oncogenic Kras-lnduced GM-CSF Production Promotes the Development of Pancreatic Neoplasia. Cancer Cell 21:836-847.
42. Revoltella, R. P., M. Menicagli, and D. Campani. 2012. Granulocyte-macrophage colony-stimulating factor as an autocrine survival-growth factor in human gliomas. Cytokine 57:347-359.
43. Rosenberg, A. S. 2006. Effects of protein aggregates: an immunologic perspective. The AAPS journal 8:E501-507.
44. Schon, M., D. Denzer, R. C. Kubitza, T. Ruzicka, and M. P. Schon. 2000. Critical role of neutrophils for the generation of psoriasiform skin lesions in flaky skin mice. J Invest Dermatol 114:976-983.
45. Schweizerhof, M., S. Stosser, M. Kurejova, C. Njoo, V. Gangadharan, N. Agarwal, M. Schmelz, K. K. Bali, C. W. Michalski, S. Brugger, A. Dickenson, D. A. Simone, and R. Kuner. 2009. Hematopoietic colony-stimulating factors mediate tumor-nerve interactions and bone cancer pain. Nat Med 15:802-807.
46. Serafini, P., R. Carbley, K. A. Noonan, G. Tan, V. Bronte, and I. Borrello. 2004. High-dose granulocyte-macrophage colony-stimulating factor-producing vaccines impair the immune response through the recruitment of myeloid suppressor cells. Cancer Res 64:6337-6343.
47. Sherlock, J. P., B. Joyce-Shaikh, S. P. Turner, C. C. Chao, M. Sathe, J. Grein, D. M. Gorman, E. P. Bowman, T. K. McClanahan, J. H. Yearley, G. Eberl, C. D. Buckley, R. A. Kastelein, R. H. Pierce, D. M. Laface, and D. J. Cua. 2012. IL-23 induces spondyloarthropathy by acting on ROR-gammat(+) CD3(+)CD4(−)CD8(−) entheseal resident T cells. Nat Med 18:1069-1076.
48. Shoenfeld, Y., S. C. Hsu-Lin, J. E. Gabriels, L. E. Silberstein, B. C. Furie, B. Furie, B. D. Stollar, and R. S. Schwartz. 1982. Production of autoantibodies by human-human hybridomas. J Clin Invest 70:205-208.
49. Shoenfeld, Y., J. Rauch, H. Massicotte, S. K. Datta, J. Andre-Schwartz, B. D. Stollar, and R. S. Schwartz. 1983. Polyspecificity of monoclonal lupus autoantibodies produced by human-human hybridomas. N Engl J Med 308:414-420.
50. Tanaka, N., J. Watanabe, T. Kitamura, Y. Yamada, S. Kanegasaki, and K. Nakata. 1999. Lungs of patients with idiopathic pulmonary alveolar proteinosis express a factor which neutralizes granulocyte-macrophage colony stimulating factor. FEBS Lett 442:246-250.
51. Thomson, C. A., Y. Wang, L. M. Jackson, M. Olson, W. Wang, A. Liavonchanka, L. Keleta, V. Silva, S. Diederich, R. B. Jones, J. Gubbay, J. Pasick, M. Petric, F. Jean, V. G. Allen, E. G. Brown, J. M. Rini, and J. W. Schrader. 2012. Pandemic H1N1 Influenza Infection and Vaccination in Humans Induces Cross-Protective Antibodies that Target the Hemagglutinin Stem. Frontiers in immunology 3:87.
52. Uchida, K., K. Nakata, B. C. Trapnell, T. Terakawa, E. Hamano, A. Mikami, I. Matsushita, J. F. Seymour, M. Oh-Eda, I. Ishige, Y. Eishi, T. Kitamura, Y. Yamada, K. Hanaoka, and N. Keicho. 2003. High affinity autoantibodies specifically eliminate granulocyte-macrophage colony-stimulating factor activity in the lungs of patients with idiopathic pulmonary alveolar proteinosis. Blood.
53. Wang, F., S. Sen, Y. Zhang, I. Ahmad, X. Zhu, I. A. Wilson, V. V.

Smider, T. J. Magliery, and P. G. Schultz. 2013. Somatic hypermutation maintains antibody thermodynamic stability during affinity maturation. Proc Natl Acad Sci USA 110:4261-4266.
54. Wang, Y., C. A. Thomson, L. L. Allan, L. M. Jackson, M. Olson, T. R. Hercus, T. L. Nero, A. Turner, M. W. Parker, A. L. Lopez, T. K. Waddell, G. P. Anderson, J. A. Hamilton, and J. W. Schrader. 2013. Characterization of pathogenic human monoclonal autoantibodies against GM-CSF. Proc Natl Acad Sci USA 110:7832-7837.
55. Wardemann, H., S. Yurasov, A. Schaefer, J. W. Young, E. Meffre, and M. C. Nussenzweig. 2003. Predominant autoantibody production by early human B cell precursors. Science 301:1374-1377.
56. Wen, L., M. Hanvanich, C. Werner-Favre, N. Brouwers, L. H. Perrin, and R. H. Zubler. 1987. Limiting dilution assay for human B cells based on their activation by mutant EL4 thymoma cells: total and antimalaria responder B cell frequencies. Eur J Immunol 17:887-892.
57. Williamson, D. J., C. G. Begley, M. A. Vadas, and D. Metcalf. 1988. The detection and initial characterization of colony-stimulating factors in synovial fluid. Clin Exp Immunol 72:67-73.
58. Wu, H., D. S. Pfarr, S. Johnson, Y. A. Brewah, R. M. Woods, N. K. Patel, W. I. White, J. F. Young, and P. A. Kiener. 2007. Development of motavizumab, an ultra-potent antibody for the prevention of respiratory syncytial virus infection in the upper and lower respiratory tract. J Mol Biol 368:652-665.
59. Zvaifler, N. J., and G. S. Firestein. 1988. Cytokines in chronic inflammatory synovitis. Scand J Rheumatol Suppl 76:203-210.

1. An isolated antibody or antibody fragment that neutralizes the bioactivity of GM-CSF comprising the heavy chain CDR sequences of SEQ ID NOS:1, 2 and 3 and/or functional variants thereof or the light chain CDR sequences of SEQ ID NOS:7, 8 and 9 or functional variants thereof.

2. The isolated antibody or antibody fragment of claim 1, comprising the heavy chain CDR sequences of SEQ ID NOS:1, 2 and 3 or functional variants thereof and the light chain CDR sequences of SEQ ID NOS:7, 8 and 9 or functional variants thereof.

3. The isolated antibody or antibody fragment of claim 1 further comprising a phenylalanine as the first amino acid of framework region 3 after the CDR2 of the heavy chain.

4. The isolated antibody or antibody fragment of claim 1 further comprising a threonine as the first amino acid of framework region 3 after the CDR2 of the light chain.

5. An isolated antibody or antibody fragment that neutralizes the bioactivity of GM-CSF comprising the heavy chain variable region as shown in the amino acid sequence of SEQ ID NO:13 or 21 or a functional variant thereof and/or the light chain variable region as shown in the amino acid sequence of SEQ ID NO:14 or 22 or a functional variant thereof.

6. The isolated antibody or antibody fragment of claim 5 comprising the heavy chain variable region as shown in the amino acid sequence of SEQ ID NO:13 or 21 or a functional variant thereof and the light chain variable region as shown in the amino acid sequence of SEQ ID NO:14 or 22 or a functional variant thereof.

7. (canceled)

8. The isolated antibody, or an antibody fragment thereof of claim 1, wherein the said antibody, or an antibody fragment inhibits GM-CSF in the TF-1 assay with an IC50 of less than 3 ng/mL.

9. The isolated antibody, or an antibody fragment thereof of claim 1, wherein the antibody or antibody fragment competes for binding to GM-CSF with an antibody having the heavy chain variable region as shown in SEQ ID NO:13 or 21 and the light chain variable region as shown in SEQ ID NO:14 or 22.

10. An isolated nucleic acid molecule encoding the heavy or light chain variable regions of the isolated antibody or antibody fragment thereof of claim 1.

11. An expression vector comprising the nucleic acid molecule of claim 10 operatively linked to suitable regulatory sequences.

12. An expression vector comprising a nucleic acid encoding the heavy chain variable region of the isolated antibody or antibody fragment thereof of claim 1 and an expression vector comprising a nucleic acid encoding the light chain variable region of the isolated antibody or antibody fragment of claim 1, operatively linked to suitable regulatory sequences.

13. (canceled)

14. A pharmaceutical composition comprising the antibody or antibody fragment of claim 1, and a pharmaceutically acceptable carrier.

15. A method for suppressing an immune response in a subject in need thereof comprising administering the antibody or antibody fragment of claim 1 to the subject.

16. The method of claim 15, for treating inflammation or an inflammatory disease.

17. The method of claim 16, wherein the inflammatory disease is arthritis, multiple sclerosis, asthma, chronic obstructive pulmonary disease (COPD), ankylosing spondylitis or psoriasis.

18. The method of claim 15, for treating cancer associated with myeloid-derived suppressor cells that suppress the adaptive immune response against cancer cells.

19. The method of claim 18, wherein the cancer is renal, colon, prostate, ovarian, melanoma, glioma, pancreatic or cervical cancer.

20. The method of claim 15, for treating pain caused by cancer or osteoarthritis.

21. A method for treating bone or cartilage loss, pain, and/or juvenile myelomonocytic leukemia in a subject in need thereof comprising administering the antibody or antibody fragment of claim 1 to the subject.

22. The method of claim 15, wherein the subject is a human.

23. The method of claim 20, wherein the subject is human.

24. The method of claim 21, wherein the subject is human.
